(19) United States
(12) Patent Application Publication
(10) Pub. No.: US 2005/0096389 A1
(43) Pub. Date: May 5, 2005

(54) Polyol ester compounds useful in preparation of a catalyst for olefins polymerization, process for preparing the same and use thereof

(75) Inventors: Mingzhi Gao, Beijing (CN); Jun Wang, Beijing (CN); Changxiu Li, Beijing (CN); Jiyu Li, Beijing (CN); Tianyi Li, Beijing (CN); Xianzhong Li, Beijing (CN); Jing Ma, Beijing (CN); Lingyan Xing, Beijing (CN); Haitao Liu, Beijing (CN)

Correspondence Address: LADAS & PARRY, 26 WEST 61ST STREET, NEW YORK, NY, 10023, US

(73) Assignee: CHINA PETROLEUM & CHEMICAL, Beijing (CN)

(21) Appl. No.: 10/503,119

(22) PCT Filed: Jan. 30, 2003
(86) PCT No.: PCT/CN03/00111

(30) Foreign Application Priority Data
Feb. 7, 2002 (CN) 02100896.5

(51) Int. Cl. A61K 31/225

(52) U.S. Cl. 514/548; 554/227; 554/228

(57) ABSTRACT

The present application relates to polyol ester compounds, having general formula (I): R₁CO—O—CR₃R4-A-CR₅R₆—O—CO—R₂ (I) wherein, R, and R₂ groups, which may be identical or different, can be substituted or unsubstituted hydrocarbyl having 1 to 20 carbon atoms, R₃-R₆ groups, which may be identical or different, can be selected from the group consisting of hydrogen, halogen or substituted or unsubstituted hydrocarbyl having 1 to 20 carbon atoms, R₁-R₆ groups optionally contain one or more hetero-atoms replacing carbon, hydrogen atom or the both, said hetero-atom is selected from the group consisting of nitrogen, oxygen, sulfur, silicon, phosphorus and halogen atom, two or more of R₃-R₆ groups can be linked to form saturated or unsaturated monocyclic or polycyclic ring; A is a single bond or bivalent linking group with chain length between two free radicals being 1-10 atoms, wherein said bivalent linking group is selected from the group consisting of aliphatic, alicyclic and aromatic bivalent radicals, and can carry C₁-C₂₀linear or branched substituents; one or more of carbon atom and/or hydrogen atom on the substituents can be replaced by a hetero-atom selected from the group consisting of nitrogen, oxygen, sulfur, silicon, phosphorus, and halogen atom, and two or more said substituents on the linking group as well as above-mentioned R₃-R₆ groups can be linked to form saturated or unsaturated monocyclic or polycyclic ring. The compounds of formula (I) find use in preparing a catalyst for olefin polymerization.

### CROSS REFERENCE OF RELATED APPLICATION

The present application claims priority based on Chinese Patent Application No. 02100896.5, filed on Feb. 7, 2002, which is incorporated herein by reference in its entirety and for all purposes.

### TECHNICAL FIELD

The present invention relates to a type of novel compounds, a process for preparing them and their use in preparing a catalyst for olefin polymerization, in particular, to a compound comprising two or more ester radicals, a process for preparing them and their use in preparing a catalyst for olefin polymerization.

### TECHNICAL BACKGROUND

It is well known that solid titanium catalyst component with magnesium, titanium, halogen and electron donor as basic compositions can be used in the polymerization of olefin CH₂═CHR, especially in the polymerization of alpha-olefins having 3 or more carbon atoms, higher isotactic polymer can be obtained in higher yield. An electron donor compound is one of indispensable compositions of catalyst component, and with the development of internal donor compound, polyolefin catalyst is continuously renovated. At present, a large amount of various electron donor compounds have been disclosed, for instance, polycarboxylic acids, monocarboxylic esters or polycarboxylic esters, anhydrides, ketones, monoethers or polyethers, alcohols, amines, and their derivatives, among of which aromatic dicarboxylic ester, such as di-n-butyl phthalate or diisobutyl phthalate (cf. U.S. Pat. No. 4,784,983), is common.

In recent years, the use of other compounds as electron donor compounds of catalyst for polymerization of olefins have been tried, for examples, U.S. Pat. No. 4,971,937 and EP 0728769 disclosed a catalyst component for polymerization of olefins, which used special 1,3-diether compounds containing two ether groups, such as 2-isoamyl-2-isopropyl-1,3-dimethoxypropane, 2,2-diisobutyl-1,3-dimethoxypropane and 9,9-bis(methoxymethyl)fluorene and the like as electron donor.

Lately, a special type of aliphatic dicarboxylic ester compounds, such as succinate, malonate, glutarate and the like had been disclosed (cf. WO98/56830, WO98/56834, WO01/57099, WO01/63231 and WO00/55215), and the use of said electron donor compound not only enhanced the activity of catalyst but also substantially broadened the distribution of the molecular weight of the propylene polymer obtained.

However, above-mentioned olefin polymerization catalysts prepared utilizing disclosed aromatic dicarboxylic ester compound, 1,3-diether compound containing two ether groups and aliphatic dicarboxylic ester compound exist certain defects in actual use. For instance, the catalytic activity of the catalyst with aromatic dicarboxylic ester compound is low, and the distribution of the molecular weight of the polymer obtained is narrow; although the catalyst with 1,3-diether compound has high catalytic activity and good hydrogen response, the distribution of the molecular weight of the polymer obtained is narrow, and this is disadvantageous in the development of different grades of polymers; and the catalytic activity of the catalyst with aliphatic dicarboxylic ester compound disclosed recently is still some low, and when external donor compound is not used, the isotacticity of the polymer obtained is lower.

The inventors have surprisingly found that an olefin polymerization catalyst with excellent general properties can be obtained by using a polyol ester compound with a special structure as electron donor. When the catalyst is used in the polymerization of propylene, satisfactory polymerization yield can be obtained, and the stereo-direction of the polymer is very high. Even if an external donor is not used, relatively high isotactic polymer can still be obtained. Meanwhile, the hydrogen response of the catalyst is excellent, and the distribution of the molecular weight of the polymer obtained is relatively wide, and these properties are desirable in the development of different grades of polymers. In addition, when the catalyst is used in the copolymerization of olefins, especially in the copolymerization of ethylene and propylene, less gel content can be achieved, therefore, it has better copolymerization property.

### DESCRIPTION OF THE INVENTION

One object of the present invention is to provide a polyol ester compound having a general formula (I):
R₁CO—O—CR₃R₄-A-CR₅R₆—O—CO—R₂ (I)
wherein, R₁ and R₂ groups, which may be identical or different, can be substituted or unsubstituted hydrocarbyl having 1 to 20 carbon atoms, R₃-R₆ groups, which may be identical or different, can be selected from the group consisting of hydrogen, halogen or substituted or unsubstituted hydrocarbyl having 1 to 20 carbon atoms, R₁-R₆ groups optionally contain one or more hetero-atoms replacing carbon, hydrogen atom or the both, said hetero-atom is selected from the group consisting of nitrogen, oxygen, sulfur, silicon, phosphorus and halogen atom, two or more of R₃-R₆ groups can be linked to form saturated or unsaturated monocyclic or polycyclic ring;
A is a single bond or bivalent linking group with chain length between two free radicals being 1-10 atoms, wherein said bivalent linking group is selected from the group consisting of aliphatic, alicyclic and aromatic bivalent radicals, and can carry C₁-C₂₀ linear or branched substituents; one or more of carbon atom and/or hydrogen atom on the substituents can be replaced by a hetero-atom selected from the group consisting of nitrogen, oxygen, sulfur, silicon, phosphorus, and halogen atom, and two or more said substituents on the linking group as well as above-mentioned R₃-R₆ groups can be linked to form saturated or unsaturated monocyclic or polycyclic ring.

As used herein, the term “hydrocarbyl” intend to include linear of branched aliphatic radical, such as alkyl, alkenyl, and alkynyl; alicyclic radical, such as cycloalkyl, cycloalkenyl; aromatic radical, such as aryl, fused ring aryl, and combination thereof, such as alkaryl, and aralkyl.

In a preferred embodiment of the present invention, the polyol ester compounds of general formula (I) are 1,2-diol ester compounds of general formula (II): 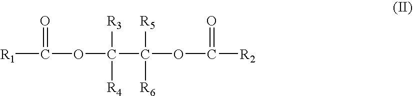
wherein R₁-R₆ have the meanings as defined in general formula (I), with the proviso that R₃, R₄, R₅, and R₆ are not hydrogen simultaneously, and at least one of R₁ and R₂ is a group containing a phenyl ring. Preferably, one group of R₃ and R₄, R₅ and R₆ in the formula (II), respectively, is hydrogen, and the other is selected from the group consisting of methyl, ethyl, propyl, isopropyl, butyl, tert-butyl, phenyl, and halophenyl group. Preferably, at least one group of R₁ and R₂ is phenyl or phenyl substituted by halogen or alkyl having 1 to 20 carbon atoms. Most preferably, both R₁ and R₂ are phenyl or phenyl substituted by halogen or alkyl having 1 to 20 carbon atoms.

Examples of the compounds of general formula (II) include, but not limited to:
1,2-ethylene glycol dibenzonate,
1,2-butandiol dibenzonate,
2,3-butandiol dibenzonate.

In another preferred embodiment of the present invention, the polyol ester compounds of general formula (I) are 1,3-diol ester compounds of general formula (III): 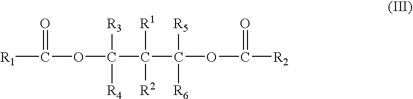
wherein R₁-R₆ have the meanings as defined in general formula (I), R¹ and R² are independently each other hydrogen or C₁-C₂₀ hydrocarbyl group, with the proviso that R¹, R², R₃, R₄, R₅, and R₆ are not hydrogen simultaneously, and can not be linked to form a ring.

Preferably, the polyol ester compounds of general formula (I) are 1,3-diol ester compounds of general formula (III), wherein when R₃, R₄, R₅ and R₆ are hydrogen, R¹ and R² are independently selected from C₃-C₂₀ alkyl, cycloalkyl, aryl, alkaryl and aralkyl, such as propyl, butyl, and the like.

Preferably, the polyol ester compounds of general formula (I) are 1,3-diol ester compounds of general formula (III), wherein when one group of R₃ and R₄, R₅ and R₆, respectively, is hydrogen and the other is methyl, and R¹ and R² are hydrogen simultaneously or hydrogen and methyl respectively, at least one of R₁ and R₂ is a group containing a phenyl ring substituted by halogen or alkyl on ortho- or meta-position.

Preferably, the polyol ester compounds of general formula (I) are 1,3-diol ester compounds of general formula (III), wherein one group of R₃ and R₄, R₅ and R₆, respectively, is hydrogen, and the other is ethyl, propyl, isopropyl, butyl, tert-butyl, phenyl, or halophenyl group, with the proviso that the groups other than hydrogen can not be phenyl simultaneously; R¹ and R², which are identical or different, represent hydrogen or methyl, ethyl, propyl, isopropyl, butyl, tert-butyl, allyl, phenyl, or halophenyl group; and at least one of R₁ and R₂ is a group containing a phenyl ring. Preferably, at least one group of R₁ and R₂ is phenyl or phenyl substituted by halogen or alkyl having 1 to 20 carbon atoms. Most preferably, both R₁ and R₂ are phenyl or phenyl substituted by halogen or alkyl having 1 to 20 carbon atoms.

Examples of the compounds of general formula (III) include, but not limited to:
2,4-pentanediol di(m-chlorobenzoate),
2,4-pentanediol di(o-bromobenzoate),
2,4-pentanediol di(p-methylbenzoate),
2,4-pentanediol di(p-tert-butylbenzoate),
2,4-pentanediol di(p-butylbenzoate),
2,4-pentanediol monobenzoate monocinnamate,
2,4-pentanediol dicinnamate,
heptan-6-ene-2,4-diol dibenzoate,
3,5-heptandiol dibenzoate,
2,6-dimethyl-3,5-heptandiol dibenzoate,
6-methyl-2,4-heptanediol dibenzoate,
6-methyl-2,4-heptanediol di(p-chlorobenzoate),
6-methyl-2,4-heptanediol di(p-methylbenzoate),
6-methyl-2,4-heptanediol di(m-methylbenzoate),
6-methyl-2,4-heptanediol dipivalate,
3-methyl-2,4-pentanediol di(p-chlorobenzoate),
3-methyl-2,4-pentanediol di(p-methylbenzoate),
3-butyl-2,4-pentanediol di(p-methylbenzoate),
3-methyl-2,4-pentanediol di(p-tert-butylbenzoate),
3-methyl-2,4-pentanediol monobenzonate monocinnamate,
3,3-dimethyl-2,4-pentandiol dibenzoate,
3,3-dimethyl-2,4-pentandiol monobenzonate monocinnamate,
3-ethyl-2,4-pentandiol dibenzoate,
3-butyl-2,4-pentandiol dibenzoate,
3-allyl-2,4-pentandiol dibenzoate,
4-methyl-3,5-heptandiol dibenzoate,
2-ethyl-1,3-hexandiol dibenzoate,
2,2,4-trimethyl-1,3-pentandiol dibenzoate,
4-methyl-3,5-octandiol dibenzoate,
5-methyl-4,6-nonandiol dibenzoate,
2-methyl-1,3-diphenyl-1,3-propylene-glycol dibenzoate,
1,3-diphenyl-1,3-propylene-glycol dipropionate,
2-methyl-1,3-diphenyl-1,3-propylene-glycol dipropionate,
2-methyl 1,3-diphenyl-1,3-propylene-glycol diacetate,
2,2-dimethyl-1,3-diphenyl-1,3-propylene-glycol dibenzoate,
2,2-dimethyl-1,3-diphenyl-1,3-propylene-glycol dipropionate,
2-methyl-1-phenyl-1,3-butandiol dibenzoate,
2-methyl-1-phenyl-1,3-butandiol dipivalate,
heptan-6-ene-2,4-diol dipivalate,
2,2,4,6,6-pentamethyl-3,5-hexandiol dibenzoate,
1,3-di-tert-butyl-2-ethyl-1,3-propylene-glycol dibenzoate,
1,3-diphenyl-1,3-propylene-glycol diacetate,
2-(2-furyl)-2-methyl-1,3-butandiol dibenzoate,
1,1-di(acryloyloxymethyl)-3-cyclohexene,
2-isoamyl-2-isopropyl-1,3-propylene-glycol dibenzoate,
2-isoamyl-2-isopropyl-1,3-propylene-glycol di(p-chlorobenzoate),
2-isoamyl-2-isopropyl-1,3-propylene-glycol di(m-chlorobenzoate),
2-isoamyl-2-isopropyl-1,3-propylene-glycol di(p-methoxybenzoate),
2-isoamyl-2-isopropyl-1,3-propylene-glycol di(p-methylbenzoate),
2-isoamyl-2-isopropyl-1,3-propylene-glycol monobenzoate monopropionate,
2-isoamyl-2-isopropyl-1,3-propylene-glycol dipropionate,
2-isoamyl-2-isopropyl-1,3-propylene-glycol diacrylate,
2-isoamyl-2-isopropyl-1,3-propylene-glycol dicinnamate,
2,2-diisobutyl-1,3-propylene-glycol dibenzoate,
2-isoamyl-2-isopropyl-1,3-propylene-glycol 2,2′-biphenyl dicarboxylate,
2-isoamyl-2-isopropyl-1,3-propylene-glycol phthalate,
1,3-diisopropyl-1,3-propylene-glycol di(4-butylbenzoate),
3-methyl-1-trifluoromethyl-2,4-pentandiol dibenzoate,
1,1,1-trifluoro-3-methyl-2,4-pentandiol dibenzoate,
4,4,4-trifluoro-1-(2-naphthyl)-1,3-butandiol dibenzoate,
2-ethyl-2-methyl-1,3-propylene-glycol dipropylformate,
2,4-pentanediol di(p-fluoromethylbenzoate),
4,6-nonandiol dibenzoate,
2,4-pentandiol di(2-furancarboxylate),
2-amino-1-phenyl-1,3-propylene-glycol dibenzoate,
2,2-dimethyl-1,3-propylene-glycol dibenzoate,
3-butyl-3-methyl-2,4-pentandiol dibenzoate,
3,6-dimethyl-2,4-heptandiol dibenzoate,
2,2,6,6-tetramethyl-3,5-heptandiol dibenzoate.

In still another preferred embodiment of the present invention, the polyol ester compounds of general formula (I) are 1,4-diol ester compounds of general formula (IV): 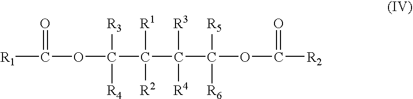
wherein R₁-R₆ have the meanings as defined in general formula (I), R¹-R⁴ are independently each other hydrogen or C₁-C₂₀ hydrocarbyl group, with the proviso that R¹-R⁴ are not hydrogen simultaneously, and R¹-R⁴ as well as R₃-R₆ can not be linked to form a ring.

Preferably, in general formula (IV), one group of R₃ and R₄, R₅ and R₆, respectively, is hydrogen, and the other is methyl, ethyl, propyl, isopropyl, butyl, tert-butyl, phenyl, or halophenyl group; R¹-R⁴, which are identical or different, represent hydrogen or methyl, ethyl, propyl, isopropyl, butyl, tert-butyl, allyl, phenyl, or halophenyl group; and at least one of R₁ and R₂ is a group containing a phenyl ring. Preferably, at least one group of R₁ and R₂ is phenyl or phenyl substituted by halogen or alkyl having 1 to 20 carbon atoms. Most preferably, both R₁ and R₂ are phenyl or phenyl substituted by halogen or alkyl having 1 to 20 carbon atoms.

Examples of the compounds of general formula (IV) include, but not limited to:
2,3-diisopropyl-1,4-butandiol dibenzoate,
2,3-dimethyl-1,4-butandiol dibenzoate,
2,3-diethyl-1,4-butandiol dibenzoate,
2,3-dibutyl-1,4-butandiol dibenzoate,
2,3-diisopropyl-1,4-butandiol dibutyrate,
2,5-hexandiol dicinnamate,
2,5-dimethyl-2,5-hexandiol dibenzoate,
2,5-dimethyl-2,5-hexandiol dipropionate,
2,5-dimethyl-hexa-3-yne-2,5-diol dibenzoate,
hexa-3-yne-2,5-diol dibenzoate (T),
hexa-3-yne-2,5-diol dibenzoate (S),
hexa-3-yne-2,5-diol di(2-furancarboxylate),
1,1-bis(benzoyloxyethyl)cyclohexane.

In still another preferred embodiment of the present invention, the polyol ester compounds of general formula (I) are 1,5-diol ester compounds of general formula (V): 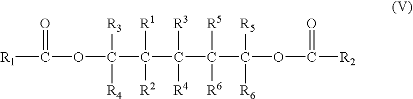
wherein R₁-R₆ have the meanings as defined in general formula (I), R¹-R⁶ are independently each other hydrogen or C₁-C₂₀ hydrocarbyl group, with the proviso that R¹-R⁶ as well as R₃-R₆ are not hydrogen simultaneously, and can not be linked to form a ring. Preferably, at least one group of R₁ and R₂ is phenyl or phenyl substituted by halogen or alkyl having 1 to 20 carbon atoms. Most preferably, both R₁ and R₂ are phenyl or phenyl substituted by halogen or alkyl having 1 to 20 carbon atoms.

Examples of the compounds of general formula (V) include, but not limited to:
2,2-dimethyl-1,5-pentanediol dibenzoate,
1,5-diphenyl-1,5-pentanediol dibenzoate,
1,5-diphenyl-1,5-pentanediol dipropionate,
2,6-dimethyl-2,6-heptanediol dibenzoate,
bis(2-benzoyloxynaphthyl)methane.

In yet another preferred embodiment of the present invention, the polyol ester compounds of general formula (I) are 1,6-diol ester compounds of general formula (VI): 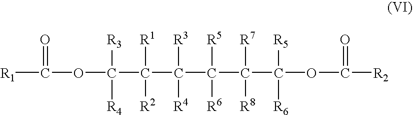
wherein R₁-R₆ have the meanings as defined in general formula (I), R¹-R⁸ are independently each other hydrogen or C₁-C₂₀ hydrocarbyl group, with the proviso that R¹-R⁸ as well as R₃-R₆ are not hydrogen simultaneously, and can not be linked to form a ring. Preferably, at least one group of R₁ and R₂ is phenyl or phenyl substituted by halogen or alkyl having 1 to 20 carbon atoms. Most preferably, both R₁ and R₂ are phenyl or phenyl substituted by halogen or alkyl having 1 to 20 carbon atoms.

Examples of the compounds of general formula (VI) include, but not limited to:
3,4-dibutyl-1,6-hexandiol dibenzoate,
2,2′-biphenyldimethanol dipivalate,
2,2′-biphenyldimethanol dibenzoate,
2,2′-biphenyldimethanol dipropionate,
2,2′-binaphthyldimethanol dibenzoate.

Another object of the present invention is to provide a process for preparing the polyol ester compounds according to the invnetion, comprising esterifying a polyol compound of general formula (VIII)
HO—CR₃R₄-A-CR₅R₆—OH (VII)
wherein A, R₃-R₆ are as defined in the formula (I),
with corresponding carboxylic acid, acyl halide or carboxylic acid anhydride.

In yet another preferred embodiment of the present invention, the polyol ester compounds of general formula (I) are compounds of general formula (VII): 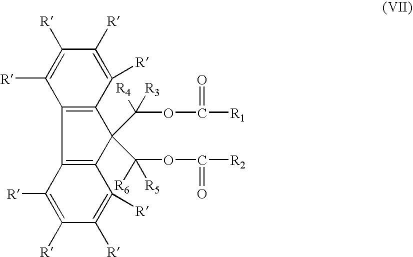
wherein R₁-R₆ are as defined in formula (I), R′, which can be identical or different, represents hydrogen, halogen atom, linear or branched C₁-C₂₀ alkyl, C₃-C₂₀ cycloalkyl, C₆-C₂₀ aryl, C₇-C₂₀ alkaryl or C₇-C₂₀ aralkyl group, with the proviso that R₁ and R₂ cannon be phenyl simultaneously.

The polyols of the formula (VII) can be synthesized by known processes in the art, for instance, references can be made on Acta Chemica Scandina-vica 21, 1967, pp. 718-720 for the synthesis of 9,9-bis(hydroxymethyl)fluorene, and CN1141285A for the method for producing dibasic alcohol.

The polyol ester compounds according to the present invention can be used as a electron donor compound in the preparation of a catalyst for olefin polymerization, and a catalyst with excellent general properties can be obtained. When the catalyst obtained is used in polymerization of propylene, satisfactory polymerization yield can be obtained, and stereo-direction of the polymer is very high. Even if an external donor is not used, relatively high isotactic polymer can still be obtained. Meanwhile, hydrogen response of the catalyst is excellent, and distribution of the molecular weight of the polymer obtained is relatively wide, and these properties are desirable in the development of different grades of polymers. In addition, when the catalyst is used in the copolymerization of olefins, especially in the copolymerization of ethylene and propylene, less gel content can be achieved, that indicates the catalyst has better copolymerization property.

### EXAMPLES

The following examples further describe the invention, but do not make limitation to the invention in any way.

Testing Methods:

1. Melting point: XT4A microscopic melting point measuring instrument (controlled temperature type).
2. Measurement of nuclear magnetic resonance: using Bruke dm×300 nuclear magnetic resonance spectrometer for ¹H-NMR (300 MHz, unless specified otherwise, solvent is CDCl₃, TMS is used as internal standard, and measuring temperature is 300K).
3. Molecular weight and molecular weight distribution (MWD) (MWD=Mw/Mn) of polymer: measured by gel permeation chromatography using PL-GPC 220 with trichlorobenzene as solvent at 150° C. (standard sample: polystyrene, flow rate: 1.0 ml/min, columns: 3×Plgel 10 um M1×ED-B 300×7.5 nm).
4. Isotacticity of polymer: measured by heptane extraction method (heptane boiling extraction for 6 hours) as the following procedure: 2 g dried polymer sample is extracted with boiling heptane in an extractor for 6 hours, then the residual substance is dried to constant weight, and the ratio of the weight of residual polymer (g) to 2 is regarded as isotacticity.

### Example 1

### Synthesis of 1,2-ethylene-glycol Dibenzoate

To 2.8 g (0.05 mol) 1,2-ethylene-glycol was added 50 ml tetrahydrofuran, then added 12.1 ml (0.15 mol) pyridine with stirring. To the resulting homogeneous mixture was slowly added 14.5 ml (0.125 mol) benzoyl chloride, and the mixture was stirred for 1 hour at room temperature, then heated refluxing for 4 hours. Upon completing the reaction, 70 ml water was added to dissolve the resulting salt. The mixture was extracted with toluene. Organic phase was separated, washed with saturated saline for two times, dried over anhydrous sodium sulfate. The solvent was removed to give a white solid. Recrystallization from ethyl acetate gave target product as a white crystal, and the yield was 92%. m.p. 69-70° C.

¹HNMR δ (ppm):4.67 (s, 4H, CH₂), 7.42-8.07 (m, 10H, ArH).

### Example 2

### Synthesis of 1,2-butandiol Dibenzoate

To the reactor were added 1,2-butandiol (2.5 g), benzoyl chloride (7.8 g), pyridine (8.8 g) and tetrahydrofuran (70 ml). The reactants was mixed and heated refluxing for 4 hours, then cooled to room temperature. Water was added to the reaction system until the inorganic phase was transparent. Organic phase was separated. Inorganic phase was extracted with ethyl ether and then the organic phase was combined. The combined organic phase was washed with water, dried over anhydrous sodium sulfate. After concentrated, 3.95 g product was separated. ¹H-NMR: δ (ppm) 1.0-1.1 (3H), 1.7-1.9 (2H), 4.4-4.6 (2H), 5.4-5.5 (1H) and 7.4-8.2 (10H).

### Example 3

### Synthesis of 2,3-butandiol Dibenzoate

Synthesis procedure was similar to that described in Example 2, and 4.4 g of product was obtained from 2,3-butandiol. ¹H-NMR: δ (ppm) 1.4-1.6 (6H), 5.3-5.5 (2H), 7.4-8.2 (10H).

### Example 4

### Synthesis of 2,4-pentanediol di(m-chlorobenzoate)

To 0.03 mol 2,4-pentanediol were added 30 ml tetrahydrofuran and 0.09 mol pyridine, then added 0.075 mol m-chlorobenzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give 2,4-pentanediol di(m-chlorobenzoate) as a colorless viscous liquid, and the yield was 95%. ¹HNMR: δ (ppm) 1.3-1.4 (6H, d, CH₃), 1.9-2.3 (2H, m, CH₂), 5.2-5.3 (2H, m, CH linked to ester radical), 7.3-8.1 (8H, m, ArH).

### Example 5

### Synthesis of 2,4-pentanediol di(o-bromobenzoate)

Synthesis procedure was similar to that described in Example 4, except that m-chlorobenzoyl chloride was replaced by o-bromobenzoyl chloride. 2,4-pentanediol di(o-bromobenzoate) as a colorless liquid was obtained at yield of 89%. ¹HNMR: δ (ppm) 1.3-1.4 (6H, m, CH₃), 2.06-2.09 (2H, d, CH₂), 5.2-5.3 (2H, m, CH linked to ester radical), 7.3-7.9 (8H, m, ArH).

### Example 6

### Synthesis of 2,4-pentanediol di(p-methylbenzoate)

Synthesis procedure was similar to that described in Example 4, except that m-chlorobenzoyl chloride was replaced by p-methylbenzoyl chloride. 2,4-pentanediol di(p-methylbenzoate) as a colorless liquid was obtained at yield of 85%. ¹HNMR: δ (ppm) 1.3-1.4 (6H, d, CH₃), 2.0-2.1 (2H, t, CH₂), 2.3-2.4 (6H, m, CH₃), 5.2-5.3 (2H, m, CH linked to ester radical), 7.1-8.0 (8H, m, ArH).

### Example 7

### Synthesis of 2,4-pentanediol di(p-tert-butylbenzoate)

Synthesis procedure was similar to that described in Example 4, except that m-chlorobenzoyl chloride was replaced by p-tert-butylbenzoyl chloride. 2,4-pentanediol di(p-tert-butylbenzoate) as a colorless liquid was obtained at yield of 80%. ¹HNMR: δ (ppm) 1.1-1.4 (24H, m, CH₃), 2.0-2.1 (2H, m, CH₂), 5.2-5.4 (2H, m, CH linked to ester radical), 7.4-8.1 (8H, m, ArH).

### Example 8

### Synthesis of 2,4-pentanediol di(p-n-butylbenzoate)

Synthesis procedure was similar to that described in Example 4, except that m-chlorobenzoyl chloride was replaced by p-n-butylbenzoyl chloride. 2,4-pentanediol di(p-n-butylbenzoate) as a colorless liquid was obtained at a yield of 91%. ¹HNMR: δ (ppm) 0.91-0.98 (6H, m, CH₃ of butyl), 1.3-1.4 (8H, m, CH₂ of butyl), 1.5-1.6 (6H, m, CH₃), 2.0-2.1 (2H, t, CH₂), 2.6-2.7 (4H, t, CH₂of butyl), 5.2-5.3 (2H, m, CH linked to ester radical), 7.1-8.0 (8H, m, ArH).

### Example 9

### Synthesis of 2,4-pentanediol Monobenzoate Monocinnamate

To 0.03 mol 2,4-pentanediol were added 30 ml tetrahydrofuran and 0.04 mol pyridine, then added 0.03 mol benzoyl chloride with stirring. The reaction was heated refluxing for 4 hours, cooled. Then to the reaction were added 20 ml tetrahydrofuran and 0.05 mol pyridine, followed by 0.04 mol cinnamyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give 2,4-pentanediol monobenzoate monocinnamate as a colorless liquid. The yield was 89%. ¹HNMR: δ (ppm) 0.8-1.4 (8H, m, CH₃), 1.9-2.1 (1H, m, CH), 5.1-5.3 (2H, m, CH linked to ester radical), 6.2-8.0 (12H, m, ArH and ═CH—).

### Example 10

### Synthesis of 2,4-pentanediol Dicinnamate

Synthesis procedure was similar to that described in Example 4, except that m-chlorobenzoyl chloride was replaced by cinnamyl chloride. 2,4-pentanediol dicinnamate as a colorless viscous liquid was obtained at a yield of 88%. ¹HNMR: δ (ppm) 1.2-1.3 (6H, m, CH₃), 2.0-2.1 (2H, d, CH₂), 5.1-5.2 (2H, m, CH linked to ester radical), 6.3-7.6 (14H, m, ArH and ═CH—).

### Example 11

### Synthesis of hepta-6-ene-2,4-diol Dibenzoate

In N₂ atmosphere free of water and oxygen, to a reactor were added in succession 0.02 mol 2,4-dihydroxy-6-heptene, 20 ml THF, and 0.06 mol pyridine. Then to the reaction mixture was added slowly dropwise 0.05 mol benzoyl chloride. Upon completing the addition, the reaction was heated refluxing for 8 hours, and react at room temperature for further 12 hours. Then the reaction mixture was filtered, and filter cake was washed with anhydrous ethyl ether for three times. The filtrate was washed with saturated saline completely, dried over anhydrous sodium sulfate. Removing solvent gave 5.1 g product. ¹HNMR: δ (ppm) 7.8 (10H, ArH), 5.6 (H, ═CH—), 5.1 (2H, CH), 4.8 (2H, ═CH₂), 2.2 (2H, CH₂), 1.7 (2H, CH₂), 1.2 (3H, CH₃).

### Example 12

### Synthesis of 3,5-heptandiol Dibenzoate

### (1) Synthesis of 3,5-heptandiol

To a mixture of 2.5 g sodium borohydride, 0.05 g sodium hydroxide, and 25 ml water, was added dropwise a solution of 14.2 g 3,5-heptandione in 30 ml methanol at 0-10° C. Upon completion, the solvent was removed by reduced pressure distillation, and the residue was continuously extracted with 40 ml ethyl acetate for 15 hours. The solvent was removed to give 3,5-heptandiol as a white solid with the yield 90%, m.p. 60-65° C. IR spectrum had a strong absorption peak at 3400 cm⁻¹, but had no absorption peak at about 1700 cm⁻¹. This demonstrated that the reduction reaction was carried out completely.

### (2) Synthesis of 3,5-heptandiol Dibenzoate

To 0.03 mol 3,5-heptandiol were added 30 ml tetrahydrofuran and 0.09 mol pyridine, then added 0.075 mol benzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give 3,5-heptandiol dibenzoate as a colorless viscous liquid. The yield was 92%.

¹HNMR: δ (ppm) 0.9-1.0 (6H, m, CH₃), 1.7-1.8 (4H, m, CH₂ of ethyl), 2.0-2.1 (2H, m, CH₂), 5.21-5.27 (2H, m, CH linked to ester radical), 7.3-8.1 (10H, m, ArH).

### Example 13

### Synthesis of 2,6-dimethyl-3,5-heptandiol Dibenzoate

### (1) Synthesis of 2,6-dimethyl-3,5-heptandiol

Synthesis procedure was similar to that described in Example 12(1), except that 3,5-heptandione was replaced by 2,6-dimethyl-3,5-heptandione, and finally the product was purified by distilling under reduced pressure. 2,6-dimethyl-3,5-heptandiol as a colorless liquid was obtained with a yield of 90%. IR spectrum had a strong absorption peak at 3400 cm⁻¹, but had no absorption peak at about 1700 cm⁻¹. This demonstrated that the reduction reaction was carried out completely.

### (2) Synthesis of 2,6-dimethyl-3,5-heptandiol Dibenzoate

Synthesis procedure was similar to that described in Example 12(2), and 2,6-dimethyl-3,5-heptandiol dibenzoate as a colorless liquid was obtained from 2,6-dimethyl-3,5-heptanediol with a yield of 88%.

¹HNMR: δ (ppm) 0.95-0.99 (12H, m, CH₃), 1.9-2.0 (4H, m, CH₂ and CH), 5.10-5.17 (2H, m, CH linked to ester radical), 7.2-8.0 (10H, m, ArH).

### Example 14

### Synthesis of 6-methyl-2,4-heptandiol Dibenzoate

### (1) Synthesis of 6-methyl-2,4-heptandiol

Synthesis procedure was similar to that described in Example 12(1), except that 3,5-heptandione was replaced by 6-methyl-2,4-heptandione, and finally the product was purified by distilling under reduced pressure. 6-dimethyl-2,4-heptandiol as a colorless liquid was obtained with a yield of 90%. IR spectrum had a strong absorption peak at 3400 cm⁻¹, but had no absorption peak at about 1700 cm⁻¹. This demonstrated that the reduction reaction was carried out completely.

### (2) Synthesis of 6-methyl-2,4-heptandiol Dibenzoate

Synthesis procedure was similar to that described in Example 12(2), except that 3,5-heptanediol was replaced by 6-methyl-2,4-heptanediol. Finally, 6-methyl-2,4-heptandiol dibenzoate as a colorless liquid was obtained with a yield of 95%. ¹HNMR: δ (ppm) 1.42-1.43 (3H, d, CH₃), 1.68 (6H, s, CH₃), 2.2-2.7 (2H, d, CH₂), 5.53-5.58 (2H, m, CH linked to ester radical), 7.3-8.0 (10H, m, ArH).

### Example 15

### Synthesis of 6-methyl-2,4-heptandiol di(p-chlorobenzoate)

Synthesis procedure was similar to that described in Example 14, except that benzoyl chloride was replaced by p-chlorobenzoyl chloride. Finally, 6-methyl-2,4-heptandiol di(p-chlorobenzoate) as a colorless liquid was obtained at a yield of 95%. ¹HNMR: δ (ppm) 1.42-1.43 (3H, d, CH₃), 1.68 (6H, s, CH₃), 2.2-2.7 (2H, d, CH₂), 5.53-5.58 (2H, m, CH linked to ester radical), 7.3-8.0 (10H, m, ArH).

δ0.8˜0.9 (3H, m, CH₃), δ1.3˜1.4 (6H, m, CH₃), δ1.4˜1.5 (2H, m, CH₂), δ1.6˜1.7 (2H, m, CH₂), δ1.8˜1.9 (1H, m, CH), δ5.3˜5.5 (2H, m, CH linked to ester radical), δ7.2˜8.0 (10H, m, ArH).

### Example 16

### Synthesis of 6-methyl-2,4-heptandiol di(p-methylbenzoate)

Synthesis procedure was similar to that described in Example 14, except that benzoyl chloride was replaced by p-methylbenzoyl chloride. Finally, 6-methyl-2,4-heptandiol di(p-methylbenzoate) as a colorless liquid was obtained at a yield of 95%. ¹HNMR: δ (ppm) δ0.8˜0.9 (6H, m, CH₃), δ1.3˜1.4 (3H, m, CH₃), δ1.4˜1.5 (2H, m, CH₂), δ1.6˜1.7 (2H, m, CH₂), δ1.8˜1.9 (1H, m, CH), δ2.3˜2.4 (6H, m, CH₃), δ5.2˜5.3 (2H, m, CH linked to ester radical), δ7.1˜7.9 (8H, m, ArH).

### Example 17

### Synthesis of 6-methyl-2,4-heptandiol di(m-methylbenzoate)

Synthesis procedure was similar to that described in Example 14, except that benzoyl chloride was replaced by m-methylbenzoyl chloride. Finally, 6-methyl-2,4-heptandiol di(m-methylbenzoate) as a colorless liquid was obtained at a yield of 95%. ¹HNMR: δ (ppm) δ0.8˜0.9 (6H, m, CH₃), δ1.3˜1.4 (3H, m, CH₃), δ1.4˜1.5 (2H, m, CH₂), δ1.6˜1.7 (2H, m, CH₂), δ1.8˜1.9 (1H, d, CH), δ2.3˜2.4 (6H, m, CH₃), δ5.2˜5.3 (2H, m, CH linked to ester radical), δ7.2˜7.9 (8H, m, ArH).

### Example 18

### Synthesis of 6-methyl-2,4-heptandiol Dipivalate

Synthesis procedure was similar to that described in Example 14, except that benzoyl chloride was replaced by pivaloyl chloride. Finally, 6-methyl-2,4-heptandiol dipivalate as a colorless liquid was obtained at a yield of 95%. ¹HNMR: δ (ppm) 0.8-0.9 (6H, d, CH₃), 1.1-1.2 (21H, m, CH₃), 1.5-1.6 (2H, m, CH₂), 4.8-5.0 (2H, m, CH linked to ester radical).

### Example 19

### Synthesis of 3-methyl-2,4-pentanediol di(p-chlorobenzoate)

(1) Synthesis of 3-methyl-2,4-pentandione

To a three-neck flask in N₂ atmosphere free of water and oxygen were successively added 0.066 mol potassium tert-butoxide and 150 ml THF. Then to the resulting mixture was slowly added dropwise 0.06 mol acetylacetone with stirring while cooling the mixture with ice-bath. Upon completing the addition, the reaction was allowed to continue at room temperature for 1 hour, then 0.07 mol iodomethane was added dropwise at room temperature. Next, the reaction was allowed to continue at room temperature for further 48 hours. After the reaction was finished, the solvent was removed by distillation. To the solid mixture was added saturated saline until the solid mixture was just completely dissolved. The solution was extracted with suitable amount of ethyl ether for three times. The organic phase was combined and dried over anhydrous sodium sulfate. The solvent was removed to give 5.8 g product.

### (2) Synthesis of 3-methyl-2,4-pentanediol

In N₂ atmosphere free of water and oxygen, to a reactor were successively added 0.024 mol LiAlH₄ and 100 ml THF, followed by adding dropwise 0.04 mol 3-methyl-2,4-pentandione while cooling the mixture with ice-bath. The reaction was allowed to continue at room temperature for 48 hours. Aqueous solution of sodium hydroxide was added carefully to stop the reaction. The reaction mixture was filtered and the cake was washed with anhydrous ethyl ether for three times. The organic phase was combined and dried over anhydrous sodium sulfate. Removing the solvent gave 3.0 g product.

### (3) Synthesis of 3-methyl-2,4-pentanediol di(p-chlorobenzoate)

Synthesis procedure was similar to that described in Example 4, and target product as a colorless liquid was obtained from 3-methyl-2,4-pentandiol and p-chlorobenzoyl chloride at a yield of 92%. ¹HNMR: δ (ppm) 1.0-1.1 (3H, m, CH₃), 1.3-1.4 (6H, m, CH₃), 1.9-2.1 (1H, m, CH), 5.1-5.3 (2H, m, CH linked to ester radical), 7.3-7.9 (8H, m, ArH).

### Example 20

### Synthesis of 3-methyl-2,4-pentanediol di(p-methylbenzoate)

Synthesis procedure was similar to that described in Example 19, except that p-chlorobenzoyl chloride was replaced by p-methylbenzoyl chloride. Finally, target product as a white solid was obtained at a yield of 92%. m.p. 91-92° C. ¹HNMR: δ (ppm) 1.1-1.2 (3H, m, CH₃), 1.3-1.4 (6H, m, CH₃), 2.1-2.2 (1H, m, CH), 2.3-2.4 (6H, m, ArCH₃), 5.2-5.3 (2H, m, CH linked to ester radical), 7.1-8.0 (8H, m, ArH).

### Example 21

### Synthesis of 3-butyl-2,4-pentanediol di(p-methylbenzoate)

To 0.03 mol 3-butyl-2,4-pentanediol were added 30 ml tetrahydrofuran and 0.09 mol pyridine, then added 0.075 mol p-methylbenzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give 3-butyl-2,4-pentanediol di(p-methylbenzoate) as a colorless liquid. The yield was 95%. ¹HNMR: δ (ppm) 0.8-0.9 (3H, m, CH₃), 1.3-1.4 (6H, m, CH₃), 1.5-1.7 (6H, m, CH₂), 1.9-2.0 (1H, m, CH), 2.3-2.4 (6H, m, ArCH₃), 5.3-5.4 (2H, m, CH linked to ester radical), δ 7.0-8.0 (8H, m, ArH).

### Example 22

### Synthesis of 3-methyl-2,4-pentanediol di(p-tert-butylbenzoate)

Synthesis procedure was similar to that described in Example 19, except that p-chlorobenzoyl chloride was replaced by p-tert-butylbenzoyl chloride. Finally, 3-methyl-2,4-pentanediol di(p-tert-butylbenzoate) as a colorless liquid was obtained, and total yield was 81% from 3-methyl-2,4-pentandione. ¹HNMR: δ (ppm) 1.1-1.4 (27H, m, CH₃), 2.0-2.1 (1H, m, CH), 5.2-5.4 (2H, m, CH linked to ester radical), 7.4-8.1 (8H, m, ArH).

### Example 23

### Synthesis of 3-methyl-2,4-pentanediol Dipivalate

In N₂ atmosphere free of water and oxygen, to a reactor were successively added 0.02 mol 3-methyl-2,4-pentanediol, 20 ml THF, and 0.06 mol pyridine, then slowly added dropwise 0.05 mol pivaloyl chloride. The reaction was heated refluxing for 8 hours, and allowed to continue at room temperature for further 12 hours. After the reaction was finished, the reaction mixture was filtered and the cake was washed with anhydrous ethyl ether for three times. The organic phase was completely washed with saturated saline, and dried over anhydrous sodium sulfate. Removing the solvent gave 4.3 g product. ¹HNMR: δ (ppm) 0.94-1.25 (27H, CH₃), 1.7 (1H, CH), 4.7-5.1 (2H, CH linked to ester radical).

### Example 24

### Synthesis of 3-methyl-2,4-pentanediol Monobenzoate Monocinnamate

Synthesis procedure was similar to that described in Example 9, and target product as a colorless viscous liquid was obtained from 3-methyl-2,4-pentanediol at a yield of 86%. ¹HNMR: δ (ppm) 0.8-1.4 (9H, m, CH₃), 1.9-2.1 (1H, m, CH), 5.1-5.3 (2H, m, CH linked to ester radical), 6.2-8.0 (12H, m, ArH and ═CH—).

### Example 25

### Synthesis of 3,3-dimethyl-2,4-pentanediol Dibenzoate

### (1) Synthesis of 3,3-dimethyl-2,4-pentandione

To 0.1 mol sodium hydride was added 100 ml anhydrous tetrahydrofuran, and slowly added dropwise 0.12 mol 3-methyl-2,4-pentandione at room temperature. Upon completion, the mixture was stirred for 0.5 hours, then 0.12 mol iodomethane was slowly added dropwise. After stirred at room temperature for 10 hours, 20 ml water was added to dissolve solid. The mixture was extracted with ethyl acetate. The solvent was removed. Distillation was carried out under reduced pressure, and cut fraction 82-84° C./1 kPa was collected. The yield was 98%.

### (2) Synthesis of 3,3-dimethyl-2,4-pentanediol

To mixture of 2.5 g sodium borohydride, 0.05 g sodium hydroxide, and 25 ml water, was added dropwise solution of 10 g 3,3-dimethyl-2,4-pentandione in 30 ml methanol at 0-10° C. Upon completion, the solvent was removed by reduced pressure distillation, and the residue was continuously extracted with 40 ml ethyl acetate for 15 hours. The solvent was removed, then distillation under reduced pressure gave 3,3-dimethyl-2,4-pentandiol as a colorless liquid. The yield was 90%. IR spectrum had a strong absorption peak at 3400 cm⁻¹, but had no absorption peak at about 1700 cm⁻¹. This demonstrated that the reduction reaction was carried out completely.

### (3) Synthesis of 3,3-dimethyl-2,4-pentanediol Dibenzoate

To 0.03 mol 3,3-dimethyl-2,4-pentanediol were added 30 ml tetrahydrofuran and 0.09 mol pyridine, then added 0.075 mol benzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give target product as a colorless liquid. The yield was 95%. ¹HNMR: δ (ppm) 1.1-1.2 (6H, m, CH₃), 1.3-1.4 (6H, m, CH₃), 5.2-5.3 (2H, m, CH linked to ester radical), 7.4-8.1 (10H, m, ArH).

### Example 26

### Synthesis of 3,3-dimethyl-2,4-pentanediol Monobenzoate Monocinnamate

Synthesis procedure was similar to that described in Example 9, and target product as a colorless viscous liquid was obtained from 3,3-dimethyl-2,4-pentanediol at a yield of 88%. ¹HNMR: δ (ppm) 1.0-1.1 (6H, m, CH₃), 1.2-1.3 (6H, m, CH₃), 5.0-5.2 (2H, m, CH linked to ester radical), 6.3-8.0 (12H, m, ArH and ═CH—).

### Example 27

### Synthesis of 3-ethyl-2,4-pentanediol Dibenzoate

### (1) Synthesis of 3-ethyl-2,4-pentandione

To a three-neck flask in N₂ atmosphere free of water and oxygen were successively added 0.066 mol potassium tert-butoxide and 150 ml THF. Then to the resulting mixture was slowly added dropwise 0.06 mol acetylacetone with stirring while cooling the mixture with ice-bath. The reaction was allowed to continue at room temperature for 1 hour, then 0.07 mol iodoethane was added dropwise at room temperature. Next, the reaction was allowed to continue at room temperature for further 48 hours. After the reaction was finished, the solvent was removed by distillation. To the solid mixture was added saturated saline until the solid mixture was just completely dissolved. The solution was extracted with suitable amount of anhydrous ethyl ether for three times. The organic phase was combined and dried over anhydrous sodium sulfate. The solvent was removed to give 6.5 g product.

### (2) Synthesis of 3-ethyl-2,4-pentanediol

In N₂ atmosphere free of water and oxygen, to a reactor were successively added 0.024 mol LiAlH₄ and 100 ml THF, followed by adding dropwise 0.04 mol 3-ethyl-2,4-pentandione while cooling the mixture with ice-bath. The reaction was allowed to continue at room temperature for 48 hours. Aqueous solution of sodium hydroxide was added carefully to stop the reaction. The reaction mixture was filtered and the cake was washed with anhydrous ethyl ether for three times. The organic phase was combined and dried over anhydrous sodium sulfate. Removing the solvent gave 3.4 g product.

### (3) Synthesis of 3-ethyl-2,4-pentanediol Dibenzoate

In N₂ atmosphere free of water and oxygen, to a reactor were successively added 0.02 mol 3-ethyl-2,4-pentanediol, 20 ml THF, and 0.06 mol pyridine, then slowly added dropwise 0.05 mol benzoyl chloride. The reaction was heated refluxing for 8 hours, and allowed to continue at room temperature for further 12 hours. After the reaction was finished, the reaction mixture was filtered and the cake was washed with anhydrous ethyl ether for three times. The organic phase was completely washed with saturated saline, and dried over anhydrous sodium sulfate. Removing the solvent gave 5.1 g product. ¹HNMR: δ (ppm) 7.25-8.17 (10H, ArH), 5.39-5.47 (2H, CH), 1.80 (1H, CH), 1.66 (2H, CH₂), 1.1-1.42 (9H, CH₃).

### Example 28

### Synthesis of 3-butyl-2,4-pentanediol Dibenzoate

Synthesis procedure was similar to that described in Example 12, and the target product as a colorless liquid was obtained from 3-butyl-2,4-pentandione at a total yield of 86%. ¹HNMR: δ (ppm) 1.1-1.2 (3H, m, CH₃), 1.3-1.4 (6H, m, CH₃), 2.0-2.1 (1H, m, CH), 5.1-5.3 (2H, m, CH linked to ester radical), 7.3-8.0 (10H, m, ArH).

### Example 29

### Synthesis of 3-allyl-2,4-pentanediol Dibenzoate

The procedure described in Example 27 was repeated, except replacing iodoethane with bromopropylene, and 5.3 grams target product was obtained. ¹HNMR: δ (ppm) 7.37-8.13 (10H, ArH), 6.0 (2H, ═CH₂), 5.38 (1H, CH), 5.12 (2H, CH), 2.49 (2H, CH₂), 2.27 (H, CH), 1.38-1.52 (6H, CH₃).

### Example 30

### Synthesis of 4-methyl-3,5-heptanediol Dibenzoate

### (1) Synthesis of 4-methyl-3,5-heptandione

To 0.02 mol sodium hydride was added 100 ml anhydrous tetrahydrofuran, slowly added dropwise 0.02 mol 3,5-heptandione at room temperature. Upon completion, the mixture was stirred for 0.5 hours, then 0.04 mol iodomethane was slowly added dropwise. After stirred at room temperature for 10 hours, 20 ml water was added. White solid was precipitated. The solid was filtered, washed with water, and dried to give 4-methyl-3,5-heptandione as a white solid, and the yield was 94%. m.p. was 91-92° C.

### (2) Synthesis of 4-methyl-3,5-heptanediol Dibenzoate

Synthesis procedure was similar to that described in Example 12, and the target product as a colorless liquid was obtained from 4-methyl-3,5-heptandione with total yield of 79%. ¹HNMR: δ (ppm) 0.9-1.0 (6H, m, CH₃), 1.1-1.2 (3H, m, CH₃), 1.7-1.8 (4H, m, CH₂ of ethyl), 2.1-2.2 (1H, m, CH₂), 5.21-5.27 (2H, m, CH linked to ester radical), 7.3-8.1 (10H, m, ArH).

### Example 31

### Synthesis of 2-ethyl-1,3-hexandiol Dibenzoate

Synthesis procedure was similar to that described in Example 4, and the target product as a colorless liquid was obtained from 2-ethyl-1,3-hexandiol and benzoyl chloride at a yield of 91%. ¹HNMR: δ (ppm) 0.9-1.1 (6H, m, CH₃), 1.4-1.6 (6H, m, CH₂), 2.2-2.3 (1H, m, CH), 4.3-4.5 (2H, m, CH₂ linked to ester radical), 5.42-5.44 (2H, m, CH linked to ester radical), 7.3-8.0 (10H, m, ArH).

### Example 32

### Synthesis of 2,2,4-trimethyl-1,3-pentanediol Dibenzoate

Synthesis procedure was similar to that described in Example 4, and the target product as a colorless viscous liquid was obtained from 2,2,4-trimethyl-1,3-pentanediol and benzoyl chloride at a yield of 85%. ¹HNMR: δ (ppm) 1.01-1.07 (6H, m, CH₃), 1.1 (6H, d, CH₃), 4.1-4.2 (2H, m, CH₂ linked to ester radical), 5.17-5.18 (1H, d, CH linked to ester radical), 7.4-8.0 (10H, m, ArH).

### Example 33

### Synthesis of 4-methyl-3,5-octanediol Dibenzoate

### (1) Synthesis of 3,5-octandione

In N₂ atmosphere free of water and oxygen, to a 3-neck flask placed in an ice-bath and equipped with addition funnel and reflux condenser were successively added 0.07 mol sodium hydride and 100 ml tetrahydrofuran. To the mixture was added dropwise a solution of 0.06 mol ethyl butyrate and 0.03 mol butanone with stirring. Upon completing the addition, the mixture was heated refluxing for 4 hours. The solvent and components with a boiling point below 110° C. were removed by distillation. To the residue was added an appropriate amount of saturated saline until the solid composition was just dissolved. The mixture was extracted with ethyl ether for three times. The organic phases were combined and dried over anhydrous sodium sulfate. The solvent was removed by distillation to give 2.4 g product.

### (2) Synthesis of 4-methyl-3,5-octanediol Dibenzoate

Synthesis procedure was similar to that described in Example 27, and the target product was obtained from 3,5-octandione. ¹HNMR: δ (ppm) 7.8 (10H, ArH), 5.28 (2H, CH), 1.8 (4H, CH₂), 1.18 (2H, CH₂), 1.0 (9H, CH₃).

### Example 34

### Synthesis of 5-methyl-4,6-nonanediol Dibenzoate

Target product was obtained according to a synthesis procedure identical with that described in Example 33, except replacing butanone with 2-pentanone.

¹HNMR: δ (ppm) 7.85 (10H, ArH), 5.38 (2H, CH), 1.7 (4H, CH₂), 1.3 (4H, CH₂), 2.45 (1H, CH), 1.0 (9H, CH₃).

### Example 35

### Synthesis of 1,3-diphenyl-2-methyl-1,3-propandiol Dibenzoate

### (1) Synthesis of 1,3-diphenyl-2-methyl-1,3-propandione

To a three-neck flask in N₂ atmosphere free of water and oxygen were successively added 0.066 mol potassium tert-butoxide and 150 ml THF. Then to the resulting mixture was slowly added dropwise 0.06 mol dibenzoylmethane with stirring while cooling the mixture with ice-bath. The reaction was allowed to continue at room temperature for 1 hour, then 0.07 mol iodomethane was added dropwise at room temperature. Next, the reaction was allowed to continue at room temperature for further 48 hours. After the reaction was finished, the solvent was removed by distillation. To the solid mixture was added saturated saline until the solid mixture was just completely dissolved. The solution was extracted with suitable amount of anhydrous ethyl ether for three times. The organic phase was combined and dried over anhydrous sodium sulfate. The solvent was removed to give 12 g product.

### (2) Synthesis of 1,3-diphenyl-2-methyl-1,3-propandiol

In N₂ atmosphere free of water and oxygen, to a reactor were successively added 0.024 mol LiAlH₄ and 100 ml THF, followed by adding dropwise 0.04 mol 1,3-diphenyl-2-methyl-1,3-propandione while cooling the mixture with ice-bath. The reaction was allowed to continue at room temperature for 48 hours. Aqueous solution of sodium hydroxide was added carefully to stop the reaction. The reaction mixture was filtered and the cake was washed with anhydrous ethyl ether for three times. The organic phase was combined and dried over anhydrous sodium sulfate. Removing the solvent gave 5.9 g target product.

### (3) Synthesis of 1,3-diphenyl-2-methyl-1,3-propandiol Dibenzoate

In N₂ atmosphere free of water and oxygen, to a reactor were successively added 0.02 mol 1,3-diphenyl-2-methyl-1,3-propandiol, 20 ml THF, and 0.06 mol pyridine, then slowly added dropwise 0.05 mol benzoyl chloride. The reaction was heated refluxing for 8 hours, and allowed to continue at room temperature for further 12 hours. After the reaction was finished, the reaction mixture was filtered and the cake was washed with anhydrous ethyl ether for three times. The organic phase was completely washed with saturated saline, and dried over anhydrous sodium sulfate. Removing the solvent gave 7.3 g target product. ¹HNMR: δ (ppm) 7.5 (20H, ArH), 5.93 (2H, CH), 1.24 (1H, CH), 0.95 (3H, CH₃).

### Example 36

### Synthesis of 1,3-diphenyl-1,3-propandiol Dipropionate

### (1) Synthesis of 1,3-diphenyl-1,3-propandiol

Synthetic process was identical with that described in Example 35(2), except replacing 1,3-diphenyl-2-methyl-1,3-propandione with dibenzoylmethane.

### (2) Synthesis of 1,3-diphenyl-1,3-propandiol Dipropionate

Synthetic procedure was identical with that described in Example 35(3), except that benzoyl chloride was replaced by propionyl chloride, and 1,3-diphenyl-2-methyl-1,3-propandiol was replaced by 1,3-diphenyl-1,3-propandiol. ¹HNMR: δ (ppm) 7.13-7.36 (10H, ArH), 5.76 (2H, CH), 2.5 (4H, CH₂), 2.11 (2H, CH₂), 1.1 (6H, CH₃).

### Example 37

### Synthesis of 1,3-diphenyl-2-methyl-1,3-propandiol Dipropionate

Synthetic procedure was identical with that described in Example 35, except that benzoyl chloride was replaced by propionyl chloride. ¹HNMR: δ (ppm) 7.25 (10H, ArH), 5.76 (2H, CH), 2.5 (4H, CH₂), 2.11 (2H, CH₂), 1.1 (6H, CH₃).

### Example 38

### Synthesis of 1,3-diphenyl-2-methyl-1,3-propandiol Diacetate

Synthetic procedure was identical with that described in Example 35, except that benzoyl chloride was replaced by acetyl chloride. ¹HNMR: δ (ppm) 7.3 (10H, ArH), 5.6 (2H, CH), 2.4 (1H, CH), 1.0 (9H, CH₃).

### Example 39

### Synthesis of 1,3-diphenyl-2,2-dimethyl-1,3-propandiol Dibenzoate

### (1) Synthesis of 1,3-diphenyl-2-methyl-1,3-propandione

Synthetic procedure was identical with that described in Example 35(1).

### (2) Synthesis of 1,3-diphenyl-2,2-dimethyl-1,3-propandione

To a three-neck flask in N₂ atmosphere free of water and oxygen were successively added 0.06 mol potassium tert-butoxide and 150 ml THF. Then to the resulting mixture was slowly added dropwise 0.05 mol 1,3-diphenyl-2-methyl-1,3-propandione with stirring while cooling the mixture with ice-bath. The reaction was allowed to continue at room temperature for 1 hour, then 0.07 mol iodomethane was added dropwise at room temperature. Next, the reaction was allowed to continue at room temperature for further 48 hours. After the reaction was finished, the solvent was removed in a rotation evaporator. To the solid mixture was added saturated saline until the solid mixture was just completely dissolved. The solution was extracted with suitable amount of anhydrous ethyl ether for three times. The organic phase was combined and dried over anhydrous sodium sulfate. The solvent was removed in a rotation evaporator, and the residue was recrystallized to give 10 g product.

### (3) Synthesis of 1,3-diphenyl-2,2-dimethyl-1,3-propandiol

Synthetic procedure was identical with that described in Example 35(2), except that 1,3-diphenyl-2-methyl-1,3-propandione was replaced by 1,3-diphenyl-2,2-dimethyl-1,3-propandione.

### (4) Synthesis of 1,3-diphenyl-2,2-dimethyl-1,3-propandiol Dibenzoate

Synthetic procedure was identical with that described in Example 35(3), and target product was obtained from 1,3-diphenyl-2,2-dimethyl-1,3-propandiol.

¹HNMR: δ (ppm): 7.3 (20H, ArH), 5.78 (2H, CH), 1.1 (6H, CH₃).

### Example 40

### Synthesis of 1,3-diphenyl-2,2-dimethyl-1,3-propandiol Dipropionate

Synthetic procedure was identical with that described in Example 39, except that benzoyl chloride was replaced by propionyl chloride. ¹HNMR: δ (ppm) 7.3 (20H, ArH), 5.89 (2H, CH), 2.4 (4H, CH₂), 0.98 (12H, CH₃).

### Example 41

### Synthesis of 1-phenyl-2-methyl-1,3-butandiol Dibenzoate

### (1) Synthesis of 1-phenyl-2-methyl-1,3-butandione

Synthetic procedure was identical with that described in Example 35(1), except that raw material dibenzoyl methane was replaced by 1-phenyl-1,3-butandione.

### (2) Synthesis of 1-phenyl-2-methyl-1,3-butandiol

Synthetic procedure was identical with that described in Example 35(2), except that reducing agent LiAlH₄ was replaced by sodium borohydride.

### (3) Synthesis of 1-phenyl-2-methyl-1,3-butandiol Dibenzoate

Synthetic procedure was identical with that described in Example 35(3).

¹HNMR: δ (ppm): 8.2 (15H, ArH), 5.6 (2H, CH), 2.1 (H, CH), 1.2 (6H, CH₃).

### Example 42

### Synthesis of 1-phenyl-2-methyl-1,3-butandiol Dipivalate

Synthetic procedure was identical with that described in Example 41, except that benzoyl chloride was replaced by pivaloyl chloride. ¹HNMR: δ (ppm) 7.3 (5H, ArH), 5.6 (2H, CH), 2.1 (H, CH), 1.2 (24H, CH₃).

### Example 43

### Synthesis of hepta-6-ene-2,4-diol Dipivalate

Synthetic procedure was identical with that described in Example 35(3), except that raw material was hepta-6-ene-2,4-diol, and benzoyl chloride was replaced by pivaloyl chloride. ¹HNMR: δ (ppm) 5.6 (1H, ═CH—), 5.1 (2H, CH), 4.8 (2H, ═CH₂), 2.2 (2H, CH₂), 1.7 (2H, CH₂), 1.2 (24H, CH₃).

### Example 44

### Synthesis of 1,3-di-tert-butyl-2-methyl-1,3-propandiol Dibenzoate

Target product was obtained according to a synthetic procedure identical with that described in Example 35, except that dibenzoyl methane was replaced by dipivaloyl methane. ¹HNMR: δ (ppm) 8.0 (10H, ArH), 5.3 (2H, CH), 2.1 (H, CH), 1.3 (18H, CH₃).

### Example 45

### Synthesis of 1,3-di-tert-butyl-2-ethyl-1,3-propandiol Dibenzoate

Target product was obtained according to a synthetic procedure identical with that described in Example 44, except that iodomethane was replaced by iodoethane. ¹H NMR (TMS, CDCl₃, ppm): 8.0 (10H, ArH), 5.3 (2H, CH), 2.1 (H, CH), 1.3 (H, CH₃).

### Example 46

### Synthesis of 1,3-diphenyl-1,3-propandiol Diacetate

### (1) Synthesis of 1,3-diphenyl-1,3-propandiol

Synthetic procedure was identical with that described in Example 35(2), except that 1,3-diphenyl-2-methyl-1,3-propandione was replaced by 1,3-diphenyl-1,3-propandione.

### (2) Synthesis of 1,3-diphenyl-1,3-propandiol Diacetate

Synthetic procedure was identical with that described in Example 35(3), except that benzoyl chloride was replaced by acetyl chloride, and 1,3-diphenyl-2-methyl-1,3-propandiol was replaced by 1,3-diphenyl-1,3-propandiol.

¹HNMR: δ (ppm) 7.13-7.35 (10H, ArH), 5.7 (2H, CH), 2.6 (2H, CH₂), 2.0 (6H, CH₃).

### Example 47

### Synthesis of 2-(2-furyl)-2-methyl-1,3-butandiol Dibenzoate

The target product was synthesized with 2-(2-furyl)-1,3-butandione as raw material according to the process described in Example 35. ¹HNMR: δ (ppm) 8.9 (3H, furan ring), 7.8 (10H, ArH), 5.1 (2H, CH), 2.15 (1H, CH), 1.0 (6H, CH₃).

### Example 48

### Synthesis of 1,1-di(propionyloxymethyl)-3-cyclohexene

Synthesis procedure was similar to that described in Example 4, and the target product as a colorless liquid was obtained from 1,1-di(hydroxymethyl)-3-cyclohexene and propionyl chloride at a yield of 92%. ¹HNMR: δ (ppm) 1.07-1.11 (6H, t, CH₃ of propionate), 1.2-1.3 (2H, t, CH₂ of cyclohexene), 2.1-2.2 (4H, m, CH₂ Of cyclohexene), 2.23-2.25 (4H, m, CH₂ Of propionate), 4.3-4.4 (4H, m, CH₂).

### Example 49

### Synthesis of 9,9-bis((m-methoxybenzoyloxy)methyl)fluorene

To 4.5 g (0.02 mol) 9,9-dihydroxymethylfluorene was added 30 ml tetrahydrofuran, then added 4.8 ml (0.06 mol) pyridine with stirring. To the resulting homogenous mixture was slowly added 6.8 ml (0.04 mol) m-methoxybenzoyl chloride. The reaction was stirred at room temperature for 1 hour, then heated refluxing for 5 hours. Upon reaction completion, 40 ml water was added to dissolve the resulting salt. The mixture was extracted with toluene. The organic phase was separated, washed with saturated saline for two times, and dried over anhydrous sodium sulfate. The solvent was removed. Recrystallization from ethyl acetate gave 9,9-bis((m-methoxybenzoyloxy)methyl)fluorene as a white crystal, the yield was 78%, and m.p. was 129-130° C. ¹HNMR: δ (ppm) 3.82 (s, 6H, CH₃O), 4.74 (m, 4H, CH₂), 6.91 (m, 4H, ArH), 7.12-7.81 (m, 16H, ArH).

### Example 50

### Synthesis of 9,9-bis((m-chlorobenzoyloxy)methyl)fluorene

To 0.03 mol 9,9-dihydroxymethylfluorene were added 30 ml tetrahydrofuran and 0.09 mol pyridine, then added 0.075 mol m-chlorobenzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by recrystallization from ethyl acetate and petroleum ether (1:1, v/v) to give target product as a white solid. The yield was 93%. ¹HNMR: δ (ppm) 4.73 (4H, s, CH₂ linked to ester radical), 7.3-8.0 (8H, m, ArH).

### Example 51

### Synthesis of 9,9-bis((p-chlorobenzoyloxy)methyl)fluorene

To 0.03 mol 9,9-dihydroxymethylfluorene were added 30 ml tetrahydrofuran and 0.09 mol pyridine, then added 0.075 mol p-chlorobenzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by recrystallization from ethyl acetate and petroleum ether (1:1, v/v) to give target product as a white solid. The yield was 93%. ¹HNMR: δ (ppm) 4.72 (4H, s, CH₂ linked to ester radical), 7.3-8.0 (8H, m, ArH).

### Example 52

### Synthesis of 9,9-bis(cinnamoyloxy)methyl)fluorene

To 5.6 g (0.03 mol) 9,9-dihydroxymethylfluorene were added 40 ml tetrahydrofuran and 7.3 ml (0.09 mol) pyridine, then added 12.5 g (0.075 mol) cinnamoyl chloride with stirring. The reaction was stirred at temperature for one hour, and heated refluxing for 4 hours. To the reaction mixture was added 40 ml water to dissolve the resulting salt. The reaction mixture was extracted with toluene. The organic phase was separated and then washed with saturated saline for two times. The extract was dried over anhydrous sodium sulfate, filtered. After removing solvent, the white solid crude was purified by recrystallization from ethyl acetate to give target product as a white crystal. The yield was 56%. m.p. 161-163° C. ¹HNMR: δ (ppm) 4.57 (4H, s, CH₂ linked to ester radical), 6.51 (2H, d, CH), 7.36-7.81 (20H, m, ArH and ═CH—Ar).

### Example 53

### Synthesis of 9-(benzoyloxymethyl)-9-(propionyloxymethyl)fluorene

To 4.5 g (0.02 mol) 9,9-di(hydroxymethyl)fluorene were added 30 ml tetrahydrofuran, and added 3.3 ml (0.03 mol) pyridine with stirring. To the resulting homogenous mixture was added slowly 2.3 ml (0.02 mol) benzoyl chloride, and the mixture was stirred at temperature for 1 hour, then heated refluxing for 5 hours. Next, the mixture was cooled to room temperature, and 20 ml tetrahydrofuran and 3.3 ml (0.03 mol) pyridine were added with stirring. To the resulting homogenous mixture was slowly added 1.8 ml (0.02 mol) propionyl chloride, and the mixture was stirred at room temperature for 1 hour, and heated refluxing for 5 hours. Then 30 ml water was added to dissolve the resulting salt. The mixture was extracted with toluene. The organic phase was separated, washed with saturated saline for two times, and dried over anhydrous sodium sulfate. The solvent was removed. Recrystallization from ethyl acetate gave 9-(benzoyloxymethyl)-9-(propionyloxymethyl)fluorene as a white crystal, and the yield was 79%. ¹HNMR: δ (ppm) 1.23 (t, 3H, CH₃), 2.39 (m, 2H, CH₂), 4.48 (s, 2H, COOCH₂), 4.62 (s, 2H, OCH₂ linked to benzoyl), 7.33-8.03 (m, 13H, ArH).

### Example 54

### Synthesis of 9,9-bis(propionyloxymethyl)fluorene

To 6.8 g (0.03 mol) 9,9-dihydroxymethylfluorene were added 40 ml tetrahydrofuran and 7.3 ml (0.09 mol) pyridine, then added 6.6 ml (0.075 mol) propionyl chloride with stirring. The reaction was stirred at temperature for one hour, and heated refluxing for 4 hours. To the reaction mixture was added 40 ml water to dissolve the resulting salt. The reaction mixture was extracted with toluene, and the extract was washed with saturated saline for two times, dried over anhydrous sodium sulfate, filtered. After removing solvent, the white solid crude was purified by recrystallization from ethyl acetate to give target product as a white crystal. The yield was 79%. m.p. 82-83° C. ¹HNMR: δ (ppm) 1.12 (6H, t, CH₃), 2.36 (4H, m, OCH₂), 4.38 (4H, s, CH₂ on propionyl), 7.32-7.77 (m, 8H, ArH).

### Example 55

### Synthesis of 9,9-bis(acryloyloxymethyl)fluorene

To 6.8 g (0.03 mol) 9,9-di(hydroxymethyl)fluorene was added 4.3 ml (0.06 mol) acrylic acid and 30 ml toluene. To the resulting homogenous mixture was added 0.2 ml concentrated sulfuric acid. Then the reaction mixture was heated refluxing for 7 hours, and the water resulted in the reaction process was separated by a water separator. The mixture was cooled to 70° C., neutralized to alkalinity with saturated sodium carbonate solution, and extracted with toluene. The organic phase was washed with saturated saline to neutrality and dried over anhydrous sodium sulfate. The solvent was removed. Purification by column chromatography gave 9,9-bis(acryloyloxymethyl)fluorene as a white solid, and the yield was 35%. m.p. was 73-75° C. ¹HNMR: δ (ppm) 4.48 (s, 4H, OCH₃), 5.85-6.43 (m, 6H, H on acryl), 7.32-7.78 (m, 8H, ArH).

### Example 56

### Synthesis of 2-isoamyl-2-isopropyl-1,3-propandiol Dibenzoate

### (1) Synthesis of 2-isopropyl-5-methyl-2-hexenal (cf. CN1036846C)

207 g Isovaleraldehyde and 26 ml OH⁻ type Amberlite IRA910 resin (produced by Rohm & Hass) were heated refluxing. The water produced was removed by using a water separator, and the reaction was stopped when about 26 ml water was collected. The resin was filtered. Distillation under reduced pressure gave a cut fraction 85-90° C./20 mmHg.

### (2) Synthesis of 2-isopropyl-5-methylhexanal

To 10 g 2-isopropyl-5-methyl-2-hexenal synthesized above were added 70 ml ethanol, 1 ml saturated NaHCO₃ solution and 0.25 g 10% Pd on carbon. N₂ was introduced, follwed by H₂, and the apparatus was connected with a graduated titration tube filled with H₂. The reaction was allowed to continue with stirring at room temperature and atmosphere pressure until the absorption of H₂ reached calculation value. The reaction mixture was filtered and the filtrate was used in the next step.

### (3) Synthesis of 2-isoamyl-2-isopropyl-1,3-propandiol

To the filtrate above were added a solution of 5.3 g K₂CO₃ in 13.1 ml water and 16.9 ml 60% CH₂O. The mixture was heated refluxing for 7 hours. Upon completing the reaction, ethanol was removed. The organic phase was separated and washed with hot water to neutrality. Distillation under reduced pressure gave 2-isoamyl-2-isopropyl-1,3-propandiol, and b.p. was 165° C./20 mmHg.

### (4) Synthesis of 2-isoamyl-2-isopropyl-1,3-propandiol Dibenzoate

To 9.4 g (0.05 mol) 2-isoamyl-2-isopropyl-1,3-propandiol was added 50 ml tetrahydrofuran, then added 12.1 ml (0.15 mol) pyridine with stirring. To the resulting homogenous mixture was slowly added 14.5 ml (0.125 mol) benzoyl chloride. Then the reaction was stirred at room temperature for 1 hour and heated refluxing for 4 hours. Upon completing the reaction, 70 ml water was added to dissolve the resulting salt. The mixture was extracted with ethyl acetate. The organic phase was separated, washed with saturated saline for two times, and dried over anhydrous sodium sulfate. The solvent was removed. Distilling under reduced pressure gave 2-isoamyl-2-isopropyl-1,3-propandiol dibenzoateas a pale yellow liquid, and the yield was 91%. ¹HNMR: δ (ppm) 0.88 (d, 6H, CH₃ of isoamyl), 1.05 (d, 6H, CH₃ of isopropyl), 1.24 (m, 2H, CH₂ of isoamyl), 1.27 (m, 2H, CH₂ of isoamy), 1.58 (m, 1H, CH of isoamyl), 2.04 (1H, m, CH of isopropyl), 4.42 (m, 4H, CH₂O of 1,3-propandiol), 7.38-8.02 (m, 10H, ArH).

### Example 57

### Synthesis of 2-isoamyl-2-isopropyl-1,3-propandiol di(p-chlorobenzoate)

To 0.03 mol 2-isoamyl-2-isopropyl-1,3-propandiol were added 30 ml tetrahydrofuran and 0.09 mol pyridine, then added 0.075 mol p-chlorobenzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give target product as a colorless liquid, and the yield 92%. ¹HNMR: δ (ppm) 0.86-0.88 (6H, d, CH₃), 1.01-1.04 (6H, d, CH₃), 1.2-1.3 (4H, m, CH₂), 1.54-1.57 (1H, m, CH), 2.01-2.04 (1H, m, CH), 4.3-4.4 (4H, m, CH₂ linked to ester radical), 7.2-7.9 (8H, m, ArH).

### Example 58

### Synthesis of 2-isoamyl-2-isopropyl-1,3-propandiol di(m-chlorobenzoate)

The target product was obtained according to a process described in example 57 except replacing p-chlorobenzoyl chloride with m-chlorobenzoyl chloride, and the yield was 95%. ¹HNMR: δ (ppm) 0.88-0.90 (6H, d, CH₃), 1.03-1.05 (6H, d, CH₃), 1.2-1.3 (4H, m, CH₂), 1.54-1.57 (1H, m, CH), 2.02-2.04 (1H, m, CH), 4.3-4.4 (4H, m, CH₂ linked to ester radical), 7.2-7.9 (8H, m, ArH).

### Example 59

### Synthesis of 2-isoamyl-2-isopropyl-1,3-propandiol di(p-methoxybenzoate)

To 3.8 g (0.02 mol) 2-isoamyl-2-isopropyl-1,3-propandiol was added 30 ml tetrahydrofuran, then added 4.8 ml (0.06 mol) pyridine with stirring. To the resulting homogenous mixture was slowly added 6.8 g (0.04 mol) p-methoxybenzoyl chloride. The reaction mixture was stirred at room temperature for 1 hour, then heated refluxing for 5 hours. Upon completing the reaction, 40 ml water was added to dissolve the resulting salt. The mixture was extracted with ethyl acetate. The organic phase was separated, washed with saturated saline for two times, and dried overanhydrous sodium sulfate. The solvent was removed. Distilling under reduced pressure gave 2-isoamyl-2-isopropyl-1,3-propandiol di(p-methoxybenzoate) as a colorless liquid, and the yield was 79%. ¹HNMR: δ (ppm) 0.89 (d, 6H, CH₃ of isoamyl), 0.98 (d, 6H, CH₃ of isopropyl), 1.19 (m, 2H, CH₂ of isoamyl), 1.38 (m, 2H, CH₂ Of isoamy), 1.49 (m, 1H, CH of isoamyl), 1.89 (m, 1H, CH of isopropyl), 3.84 (s, 6H, CH₃O of benzene ring), 4.34 (m, 4H, CH₂O of 1,3-propandiol), 6.91 (m, 4H, ArH), 7.96 (m, 4H, ArH).

### Example 60

### Synthesis of 2-isoamyl-2-isopropyl-1,3-propandiol di(p-methylbenzoate)

The target product was obtained according to the procedure described in Example 59, except replacing p-methyloxybenzoyl chloride with p-methylbenzoyl chloride. The yield was 88%. ¹HNMR: δ (ppm) 0.88 (d, 6H, CH₃ of isoamyl), 0.97 (d, 6H, CH₃ of isopropyl), 1.21 (m, 2H, CH₂ of isoamyl), 1.37 (m, 2H, CH₂ of isoamy), 1.47 (m, 1H, CH of isoamyl), 1.89 (m, 1H, CH of isopropyl), 2.38 (s, 6H, CH₃ of aromatic ring), 4.36 (m, 4H, CH₂O of 1,3-propandiol), 7.21 (m, 4H, ArH), 7.90 (m, 4H, ArH).

### Example 61

### Synthesis of 2-isoamyl-2-isopropyl-1,3-propandiol Monobenzoate Monopropionate

To 7.5 g (0.05 mol) 2-isoamyl-2-isopropyl-1,3-propandiol was added 50 ml tetrahydrofuran, then added 4.8 ml (0.06 mol) pyridine with stirring. To the resulting homogenous mixture was slowly added 4.6 ml (0.04 mol) benzoyl chloride. The mixture was stirred at room temperature for 1 hour and heated refluxing for 5 hours. Upon completing the reaction, the reaction mixture was cooled to room temperature. To the mixture was added 30 ml tetrahydrofuran, then added 4.8 ml (0.06 mol) pyridine with stirring. To the resulting homogenous mixture was slowly added 3.5 ml (0.04 mol) propionyl chloride. The mixture was stirred at room temperature for 1 hour, and heated refluxing for 5 hours. Upon completing the reaction, 40 ml water was added to dissolve the resulting salt. The mixture was extracted with ethyl acetate. The organic phase was separated, washed with saturated saline for two times, and dried over anhydrous sodium sulfate. The solvent was removed. Distillation under reduced pressure gave colorless liquid 2-isoamyl-2-isopropyl-1,3-propandiol monobenzoate monopropionate, and the yield was 91%. ¹HNMR: δ (ppm) 0.87 (d, 6H, CH₃ of isoamyl), 0.93 (d, 6H, CH₃ of isopropyl), 0.99 (t, 2H, CH₃ of propionyl), 1.06 (m, 4H, CH₂ of isoamy), 1.11 (m, 1H, CH of isoamyl), 1.14 (m, 1H, CH of isopropyl), 2.29 (m, 2H, CH₂O of 1,3-propandiol), 4.28 (m, 2H, CH₂O of 1,3-propandiol), 4.38 (m, 2H, CH₂ of propionyl), 7.41-8.03 (m, 5H, ArH).

### Example 62

### Synthesis of 2-isoamyl-2-isopropyl-1,3-propandiol Dipropionate

To 9.4 g (0.05 mol) 2-isoamyl-2-isopropyl-1,3-propandiol was added 50 ml tetrahydrofuran, then added 12.1 ml (0.15 mol) pyridine with stirring. To the resulting homogenous mixture was slowly added 11.0 ml (0.125 mol) propionyl chloride. The reaction mixture was stirred at room temperature for 1 hour, then heated refluxing for 4 hours. Upon completing the reaction, 70 ml water was added to dissolve the resulting salt. The mixture was extracted with ethyl acetate. The organic phase was separated, washed with saturated saline for two times, and dried over anhydrous sodium sulfate. The solvent was removed. Distilling under reduced pressure gave 2-isoamyl-2-isopropyl-1,3-propandiol dipropionate as a pale yellow liquid, and the yield was 91%. ¹HNMR: δ (ppm) 0.88 (d, 6H, CH₃ of isoamyl), 0.93 (d, 6H, CH₃ of isopropyl), 1.14 (m, 6H, CH₃ of propionyl), 1.34-1.39 (m, 4H, CH₂ of isoamy), 1.44 (m, 1H, CH of isoamyl), 1.85 (m, 1H, CH of isopropyl), 2.32 (m, 4H, CH₂O of 1,3-propandiol), 4.07 (m, 4H, CH₂ of propionyl).

### Example 63

### Synthesis of 2-isoamyl-2-isopropyl-1,3-propandiol Diacrylate

To 9.4 g (0.05 mol) 2-isoamyl-2-isopropyl-1,3-propandiol was added 7.5 ml (0.11 mol) acrylic acid and 30 ml toluene with stirring. To the resulting homogenous mixture was added 0.2 ml concentrated sulfuric acid. Then the reaction mixture was heated refluxing for 7 hours, and the water resulted in the reaction process was separated by a water separator. The mixture was cooled to 70° C., neutralized to alkalinity with saturated sodium carbonate solution, and extracted with ethyl acetate. The organic phase was washed with saturated saline to neutrality and dried over anhydrous sodium sulfate. The solvent was removed. Purification by column chromatography gave target product as a pale yelloe liquid, and the yield was 65%. ¹HNMR: δ (ppm) 0.87 (d, 6H, CH₃ of isoamyl), 0.92 (d, 6H, CH₃ of isopropyl), 1.15 (m, 2H, CH₂ of isoamy), 1.40 (m, 2H, CH₂ of isoamyl), 1.42 (m, 1H, CH of isoamyl), 1.88 (m, 1H, CH of isopropyl), 4.15 (m, 4H, CH₂O of 1,3-propandiol), 5.81-6.4 (m, 6H, H on acryl).

### Example 64

### Synthesis of 2-isoamyl-2-isopropyl-1,3-propandiol Dicinnamate

To 7.5 g (0.04 mol) 2-isoamyl-2-isopropyl-1,3-propandiol was added 50 ml tetrahydrofuran, then added 9.7 ml (0.12 mol) pyridine with stirring. To the resulting homogenous mixture was slowly added 16.7 g (0.1 mol) cinnamoyl chloride. The mixture was stirred at room temperature for 1 hour, and heated refluxing for 4 hours. Upon completing the reaction, 50 ml water was added to dissolve the resulting salt. The mixture was extracted with ethyl acetate. The organic phase was separated, washed with saturated saline for two times, and dried over anhydrous sodium sulfate. The solvent was removed. Column chromatography gave 2-isoamyl-2-isopropyl-1,3-propandiol dicinnamate as a yellow viacous liquid, and the yield was 51%. ¹HNMR: δ (ppm) 0.88 (d, 6H, CH₃ of isoamyl), 0.99 (d, 6H, CH₃ of isopropyl), 1.21 (m, 2H, CH₂ of isoamy), 1.47 (m, 2H, CH₂ of isoamyl), 1.51 (m, 1H, CH of isoamyl), 1.96 (m, 1H, CH of isopropyl), 4.26 (m, 4H, CH₂O of 1,3-propandiol), 6.45 (d, 2H, CH linked to carbonyl), 7.26-7.70 (m, 12H, ArH and ═CH—Ar).

### Example 65

### Synthesis of 2,2-diisobutyl-1,3-propandiol Dibenzoate

### (1) Synthesis of Diethyl 2,2-diisobutylmalonate

In N₂ atmosphere, to a reactor were added 100 ml ethanol and 5 g Na. After the reaction ended, to the reactor was added 16 g (0.1 mol) diethyl malonate and the mixture was stirred at room temperature for several minutes. Then 28 g (0.21 mol) isobutyl bromide was added, and the mixture was heated refluxing for 6 hours. To the reaction mixture was added 7.5 g (0.12 mol) sodium ethoxide, followedd by 14 g (0.1 mol) isobutyl bromide, and the reaction was heated refluxing for 8 hours. Upon completing the reaction, most of solvent was removed by distillation under reduced pressure. The residue was extracted with hexane. After removing hexane, distillation under reduced pressure gave diethyl 2,2-diisobutylmalonate. b.p. 145-146° C./20 mmHg.

### (2) Synthesis of 2,2-diisobutyl-1,3-propandiol

To 3 g (0.079 mol) LiAlH₄ was added 100 ml ethyl ether, then added 15.5 g (0.057 mol) diethyl 2,2-diisobutylmalonate dropwise with intensely stirring. The reaction mixture was heated refluxing for 5 hours, then poured into 100 g ice that was acidified with dilute hydrochloric acid. The mixture was extracted with ethyl ether. After removing ethyl ether from the extract, 2,2-diisobutyl-1,3-propandiol as a white solid was recrystallized from hexane, and the yield was 78%. m.p. 75-77° C.

### (3) Synthesis of 2,2-diisobutyl-1,3-propandiol Dibenzoate

To 7.5 g (0.04 mol) 2,2-diisobutyl-1,3-propandiol was added 50 ml tetrahydrofuran, then added 9.7 ml (0.12 mol) pyridine with stirring. To the resulting homogenous mixture was slowly added 11.6 ml (0.1 mol) benzoyl chloride. The mixture was stirred at room temperature for 1 hour, and heated refluxing for 5 hours. Upon completing the reaction, 40 ml water was added to dissolve the resulting salt. The mixture was extracted with ethyl acetate. The organic phase was separated, washed with saturated saline for two times, and dried over anhydrous sodium sulfate. The solvent was removed. Distillation under reduced pressure gave 2,2-diisobutyl-1,3-propandiol dibenzoate as a pale yellow liquid, the yield was 93%.

¹HNMR: δ (ppm) 0.91 (d, 12H, 1.21 (d, 4H, CH₂ of isobutyl), 2.05 (t, 2H, CH of isobutyl), 4.43 (m, 4H, CH₂O of 1,3-propandiol), 7.40-8.05 (m, 10H, ArH).

### Example 66

### Synthesis of 2,6-dimethyl-3,5-heptandiol di(4-n-butylbenzoate)

The synthetic procedure was identical with Example 4, and target product as a colorless viscous liquid was obtained from 2,6-dimethyl-3,5-heptandiol and 4-n-butylbenzoyl chloride at a yield of 88%. ¹HNMR: δ (ppm) 1.0-1.1 (18H, m, CH₃), 1.3-1.4 (4H, m, CH₂), 1.4-1.5 (4H, m, CH₂), 1.7-1.8 (2H, m, CH), 2.75-2.79 (4H, m, CH₂), 2.81-2.85 (2H, m, CH₂), 5.20-5.28 (2H, m, CH), 7.2-8.1 (8H, m, ArH).

### Example 67

### Synthesis of (1S,2S)-2-amino-1-phenyl-1,3-propandiol Dibenzoate

The synthetic procedure was identical with Example 4, and target product as a colorless liquid was obtained from (1S,2S)-2-amino-1-phenyl-1,3-propandiol and benzoyl chloride at a yield of 89%. ¹HNMR: δ (ppm) 4.1-4.2 (1H, m, CH), 4.42-4.47 (2H, m, NH₂), 4.6-4.7 (2H, m, CH₂), 6.81-6.84 (1H, m, CH), 7.2-8.0 (15H, m, ArH).

### Example 68

### Synthesis of 3-methyl-1-trifluoromethyl-2,4-pentandiol Dibenzoate

The synthetic process was identical with Example 2, and 4.3 g target product was obtained from 3-methyl-1-trifluoromethyl-2,4-pentandiol (3.4 g), benzoyl chloride (4 g), pyridine (4.5 g), and tetrahydrofuran (70 ml). ¹HNMR: b (ppm) 1.4 (6H), 2.2-2.4 (2H), 5.1-5.6 (1H), 5.8 (1H), 7.3-7.9 (10H).

### Example 69

### Synthesis of 1,1,1-trifluoro-3-methyl-2,4-pentandiol Dibenzoate

The synthetic process was identical with Example 2, and 5.2 g target product was obtained from 1,1,1-trifluoro-3-methyl-2,4-pentandiol (3.8 g), benzoyl chloride (4.5 g), pyridine (4.5 g), and tetrahydrofuran (70 ml). ¹HNMR: δ (ppm) 1.4 (3H), 2.2-2.4 (2H), 5.3-5.7 (2H), 5.8 (1H), 7.3-7.9 (10H).

### Example 70

### Synthesis of 4,4,4-trifluoro-1-(2-naphthyl)-1,3-butandiol Dibenzonate

To mixture of 25 g sodium borohydride, 5 g sodium hydroxide, and 1000 ml water, was added dropwise solution of 1.0 mol 4,4,4-trifluoro-1-(2-naphthyl)-1,3-butandione in 300 ml methanol while cooling the mixture with an ice-bath. Upon completion, the mixture was allowed to react for 4 hours at room temperature. Then methanol and water were removed, and the residue was continuously extracted with ethyl ether for 17 hours. The organic phase was separated from inorganic phase and concentrated, then the product was separated by column chromatography.

5 g the above obtained 4,4,4-trifluoro-1-(2-naphthyl)-1,3-butandiol, 4.4 ml benzoyl chloride, 5.5 g pyridine and 70 ml tetrahydrofuran were mixed, and heated refluxing for 4 hours. Then the reaction was cooled to room temperature, and water was added to the system until the inorganic phase was transparent. Inorganic phase was separated from organic phase, and extracted with ethyl ether. The combined organic phase was dried over anhydrous sodium sulfate. After concentrated, 3 g product was purified by column chromatography.

¹HNMR: δ (ppm) 1.2-1.6 (2H), 2.1-2.4 (2H), 7.4-8.3 (17H).

### Example 71

### Synthesis of 2,4-pentandiol di(p-fluoromethylbenzoate)

The synthetic process was identical with Example 2. 3.5 g target product was obtained from 2,4-pentandiol (2.1 g), p-fluoromethylbenzoyl chloride (9.2 g), pyridine (6 g), and tetrahydrofuran (70 ml). ¹HNMR: δ (ppm) 1.4 (6H), 1.9-2.2 (2H), 5.3-5.4 (2H), 7.4-8.2 (8H).

### Example 72

### Synthesis of 2,4-pentandiol di(2-furancarboxylate)

The synthetic process was identical with Example 2. 5.7 g target product was obtained from 2,4-pentandiol (4 g), 2-furancarboxylic acid chloride (9.1 g), pyridine (12 g), and tetrahydrofuran (50 ml). ¹HNMR: δ (ppm) 1.2-1.4 (6H), 1.9-2.1 (2H), 3.9-4.2 (2H), 4.6-4.8 (2H), 5.2-5.3 (2H), 6.5-7.5 (6H).

### Example 73

### Synthesis of 2-methyl-2-(2-furyl)-1,3-butandiol Dibenzoate

The target product was obtained by a process identical with that described in example 35 except replacing dibenzoylmethane with 2-(2-furyl)-1,3-butandione. ¹H-NMR (TMS, CDCl₃, ppm): 8.9 (3H, furan ring H), 7.8 (10H, ArH), 5.1 (2H, CH), 2.15 (1H, CH), 1.0 (6H, CH₃).

### Example 74

### Synthesis of 4-ethyl-3,5-heptandiol Dibenzoate

Synthesis procedure was similar to that described in Example 34. ¹HNMR: δ (ppm): 0.8 (10H, ArH), 5.3 (2H, CH), 2.0 (1H, CH), 1.9 (2H, CH₂), 1.7 (4H, CH₂), 1.0 (9H, CH₃).

### Example 75

### Synthesis of 2,2-dimethyl-1,3-propandiol Dibenzoate

The synthetic procedure was identical with Example 4, and target product as a colorless liquid was obtained from 2,2-dimethyl-1,3-propandiol and benzoyl chloride at a yield of 98%. ¹HNMR: δ (ppm) 0.93-0.97 (6H, t, CH₃), 1.54-1.59 (4H, m, CH₂), 4.3 (4H, s, CH₂), 7.4-8.0 (10H, m, ArH).

### Example 76

### Synthesis of 3-butyl-3-methyl-2,4-pentandiol Dibenzoate

### (1) Synthesis of 3-butyl-3-methyl-2,4-pentandione

To 0.1 mol sodium hydride was added 100 ml anhydrous tetrahydrofuran, then added slowly dropwise 0.1 mol 3-butyl-2,4-pentandione at room temperature. The reaction was stirred for 0.5 hours, then 0.12 mol iodomethane was added dropwise and the reaction was stirred at room temperature for further 10 hours. Upon completing the reaction, 20 ml water was added. The mixture was extracted with ethyl acetate. After removing the solvent, distillation under reduced pressure gave a cut fraction 84-86° C./4 kPa (165-166° C. at atmosphere pressure), and the yield was 94%.

### (2) Synthesis of 3-butyl-3-methyl-2,4-pentandiol

To the mixture of 2.5 g sodium borohydride, 0.05 g sodium hydroxide, and 25 ml water was added dropwise the mixture of 12 g 3-butyl-3-methyl-2,4-pentandione and 30 ml methanol at 0-10° C. Upon completing the addition, the solvent was removed by reduced pressure distillation. The reaction mixture was continuously extracted with 40 ml ethyl acetate for 15 hours. The solvent was removed. Distillation under reduced pressure gave colorless liquid 3-butyl-3-methyl-2,4-pentandiol, and the yield was 90%. IR spectrum had a strong absorption peak at 3400 cm⁻¹, and had no absorption peak at about 1700 cm⁻¹. This demonstrated that the reduction reaction was carried out completely.

### (3) Synthesis of 3-butyl-3-methyl-2,4-pentandiol Dibenzoate

To 0.03 mol 3-butyl-3-methyl-2,4-pentandiol were added 30 ml tetrahydrofuran and 0.09 mol pyridine, then added 0.075 mol benzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give target product. The yield was 95%. ¹HNMR: δ (ppm) 1.1-1.2 (3H, m, CH₃), 1.3-1.4 (6H, m, CH₃), 2.0-2.1 (2H, m, CH₂), 5.1-5.3 (2H, m, CH linked to ester radical), 7.3-8.0 (10H, m, ArH).

### Example 77

### Synthesis of 3,6-dimethyl-2,4-heptanediol Dibenzoate

### (1) Synthesis of 3,6-dimethyl-2,4-heptandione

To 0.1 mol sodium hydride was added 100 ml anhydrous tetrahydrofuran, then added slowly dropwise 0.1 mol 6-methyl-2,4-heptandione at room temperature. The reaction was stirred for 0.5 hours, then 0.12 mol iodomethane was added dropwise and the reaction was stirred at room temperature for further 10 hours. Upon completing the reaction, 20 ml water was added. The mixture was extracted with ethyl acetate. After removing the solvent, distillation under reduced pressure gave a cut fraction 88-90° C./1 kPa (165-166° C. at atmosphere pressure), and the yield was 94%.

### (2) Synthesis of 3,6-dimethyl-2,4-heptanediol

To the mixture of 2.5 g sodium borohydride, 0.05 g sodium hydroxide, and 25 ml water was added dropwise the mixture of 14.2 g 3,6-dimethyl-2,4-heptandione and 30 ml methanol at 0-10° C. Upon completing the addition, the solvent was removed by reduced pressure distillation. The reaction mixture was continuously extracted with 40 ml ethyl acetate for 15 hours. The solvent was removed. Distillation under reduced pressure gave colorless liquid 3,6-dimethyl-2,4-heptanediol, and the yield was 90%. IR spectrum had a strong absorption peak at 3400 cm⁻¹, and had no absorption peak at about 1700 cm⁻¹. This demonstrated that the reduction reaction was carried out completely.

### (3) Synthesis of 3,6-dimethyl-2,4-heptanediol Dibenzoate

To 0.03 mol 3,6-dimethyl-2,4-heptanediol were added 30 ml tetrahydrofuran and 0.09 mol pyridine, then added 0.075 mol benzoyl chloride with stirring. The reaction was heated refluxing for 4 hours, cooled and added 20 ml saturated saline. The reaction mixture was extracted with ethyl acetate, and the extract was dried over anhydrous sodium sulfate, filtered. After removing solvent, the crude was purified by column chromatography to give target product. The yield was 88%. ¹HNMR: δ (ppm) 1.42-1.43 (3H, d, CH₃), 1.68 (6H, s, CH₃), 2.2-2.7 (2H, d, CH₂), 5.53-5.58 (2H, m, CH linked to ester radical), 7.3-8.0 (10H, m, ArH).

### Example 78

### Synthesis of 2,2,6,6-tetramethyl-3,5-heptanediol Dibenzoate

The target product was obtained according to a synthetic process similar to that described in Example 35 from 2,2,6,6-tetramethyl-3,5-heptanedione. ¹HNMR: δ (ppm) 8.0 (10H, ArH), 5.3 (2H, CH), 2.0 (2H, CH₂), 1.3 (1H, CH₃).

### Example 79

### Synthesis of 2,3-diisopropyl-1,4-butandiol Dibenzoate

### (1) Synthesis of 2,3-diisopropyl-1,4-butandiol

A mixture of 5.1 g LiAlH₄ and 120 ml ethyl ether was cooled to 0° C., then a solution of 11 g diethyl 2,3-diisopropyl-1,4-succinate and 60 ml ethyl ether was added dropwise at that temperature. Upon completing the addition, the mixture was heated refluxing for 1 hour. Then the reaction mixture was cooled to 0° C. again, and 5 ml 15% solution of sodium hydroxide and 20 ml water were added dropwise. The mixture was warmed to room temperature and allowed to react for 0.5 hours. The reaction mixture was filtered, and the filtrate was washed, dried, concentrated and distilled under reduced pressure to give 8.4 g product with a yield of 76%. bp 118° C./0.1 mmHg. ¹HNMR: δ (ppm) 0.9 (14H), 1.4 (2H), 1.9 (4H), 3.7 (2H).

### (2) Synthesis of 2,3-diisopropyl-1,4-butandiol Dibenzoate

7.7 g 2,3-diisopropyl-1,4-butandiol and 100 ml THF were mixed, and to the mixture was added 12.5 g benzoyl chloride and 14 g pyridine. The mixture was heated refluxing for 4 hours. Upon completing the reaction, water was added to dissolve solid substance. The organic phase was separated, washed, dried, and concentrated to give 13.9 g product, and the yield was 87%.

¹HNMR: δ (ppm) 1.2-1.4 (14H), 2.0-2.2 (2H), 4.4-4.6 (4H), 7.3-8.2 (10H).

### Example 80

### Synthesis of 2,3-dimethyl-1,4-butandiol Dibenzoate

According to the synthetic processes described in Example 79, following substance was synthesized:

### (1) 2,3-dimethyl-1,4-butandiol

bp 95° C./0.1 mmHg; ¹HNMR: δ (ppm) 0.7-1.8 (8H), 3.2-3.8 (4H), 4.8 (2H).

### (2) 2,3-dimethyl-1,4-butandiol Dibenzoate

¹HNMR: (δ, ppm) 1.1-1.6 (8H), 5.0-5.5 (4H), 7.3-8.2 (10H).

### Example 81

### Synthesis of 2,3-diethyl-1,4-butandiol Dibenzoate

According to the synthetic processes described in Example 79, following substance was synthesized:

### (1) 2,3-diethyl-1,4-butandiol

bp 110° C./0.1 mmHg; ¹HNMR: δ (ppm) 0.7-1.9 (12H), 3.3-3.9 (4H), 4.8 (2H).

### (2) 2,3-diethyl-1,4-butandiol Dibenzoate

¹HNMR: (δ, ppm) 1.0-1.5 (10H), 2.1-2.3 (2H), 4.3-4.5 (4H), 7.3-8.1 (10H).

### Example 82

### Synthesis of 2,3-dibutyl-1,4-butandiol Dibenzoate

According to the synthetic processes described in Example 79, following substance was synthesized:

### (1) 2,3-dibutyl-1,4-butandiol

bp 144° C./0.2 mmHg; ¹HNMR: δ (ppm) 0.7-2.1 (18H), 3.2-3.9 (4H), 4.9 (2H).

### (2) 2,3-dibutyl-1,4-butandiol Dibenzoate

¹HNMR: (δ, ppm) 0.8-1.6 (18H), 2.1-2.3 (2H), 4.3-4.5 (4H), 7.4-8.1 (10H).

### Example 83

### Synthesis of 2,5-hexandiol Dicinnamate

To 2.4 g (0.02 mol) 2,5-hexandiol was added 30 ml tetrahydrofuran, then added 4.8 ml (0.06 mol) pyridine with stirring. To the resulting homogenous mixture was slowly added 8.3 (0.05 mol) cinnamoyl chloride, and the mixture was stirred at room temperature for 1 hour, then heated refluxing for 5 hours. Upon completing the reaction, 20 ml water was added to dissolve the resulting salt. The mixture was extracted with ethyl acetate. The organic phase was separated, washed with saturated saline for two times, and dried over anhydrous sodium sulfate. The solvent was removed. Column chromatography gave 2,5-hexandiol dicinnamate as a colorless viscous liquid, and the yield was 67%. ¹HNMR: δ (ppm) 1.25 (d, 6H, CH₃), 1.66 (m, 4H, CH₂), 5.08 (m, 2H, CH), 6.46 (d, 2H, ═CH—), 7.34-7.70 (m, 12H, ArH and ═CH—).

### Example 84

### Synthesis of 2,5-dimethyl-2,5-hexandiol Dibenzoate

Synthesis procedure was similar to that described in Example 4, and the target product as a colorless viscous liquid was obtained from 2,5-dimethyl-2,5-hexandiol and benzoyl chloride at a yield of 93%. ¹HNMR: δ (ppm) 1.6 (12H, s, CH₃), 2.0 (4H, s, CH₂), 7.4-8.0 (10H, m, ArH).

### Example 85

### Synthesis of hexa-3-yne-2,5-diol di(2-furancarboxylate)

Synthesis procedure was similar to that described in Example 2, and 6.5 g target product was obtained from hexa-3-yne-2,5-diol (4.3 g), 2-furancarboxylic acid chloride (10.5 g), pyridine (13 g), and tetrrahydrofuran (70 ml). ¹HNMR: δ (ppm) 1.45-1.72 (6H), 3.52 (2H), 6.4-7.7 (8H).

### Example 86

### Synthesis of 2,2-dimethyl-1,5-pentanediol Dibenzoate

### (1) Synthesis of Diethyl 2,2-dimethylglutarate

To 0.1 mol 2,2-dimethyl-glutaric acid were added 0.3 mol ethanol, 40 ml toluene, and 0.4 ml concentrated sulfuric acid with stirring. The mixture was heated refluxing, and the water produced was removed by using a water separator until the amount of the water separated reached theoretical value. After the reaction ended, the mixture was neutralized with saturated sodium carbonate solution and extracted with ethyl acetate. The upper layer solution was separated, washed with saturated saline until being neutral, and dried over anhydrous sodium sulfate. After the solvent was removed, distillation under reduced pressure gave diethyl 2,2-dimethylglutarate as a colorless liquid, and the yield was 90%. ¹HNMR: δ (ppm) 1.18 (6H, s, CH₃), 1.23-1.27 (6H, t, CH₃ of ethyl), 1.7-1.8 (2H, t, CH₂), 2.25-2.29 (2H, t, CH₂), 4.0-4.1 (4H, m, CH₂ of ethyl).

### (2) Synthesis of 2,2-dimethylpentanediol

While cooled by an ice-bath and stirred intensely, 0.05 mol diethyl 2,2-dimethyl glutarate was added dropwise slowly to a mixture of 3 g LiAlH₄ and 100 ml anhydrous ethyl ether. The mixture was heated refluxing for 5 hours, then cooled. Excess LiAlH₄ was decomposed with water. After filtering, the filtrate was extracted with ethyl ether, and the extract was dried over anhydrous sodium sulfate. The solvent was removed. Column chromatography gave 2,2-dimethylpentanediol as a colorless viscous liquid, and the yield was 75%. IR spectrum had a strong absorption peak at 3400 cm⁻¹, and had no absorption peak at about 1700 cm⁻¹. This demonstrated that the reduction reaction was carried out completely.

### (3) Synthesis of 2,2-dimethyl-1,5-pentanediol Dibenzoate

Synthesis procedure was similar to that described in Example 16, and the target product as a colorless viscous liquid was obtained from 2,2-dimethylpentanediol at a yield of 93%. ¹HNMR: δ (ppm) 1.0 (6H, s, CH₃), 1.3-1.4 (2H, t, CH₂), 1.6-1.7 (2H, m, CH₂), 4.0-4.3 (4H, m, CH₂ linked to ester radical), 7.4-8.1 (10H, m, ArH).

### Example 87

### Synthesis of 1,1-bis(benzoyloxyethyl)cyclohexane

### (1) 1,1-bis((ethyloxycarbonyl)methyl)cyclohexane

Synthesis procedure was similar to that described in Example 85(1), and 1,1-bis((ethyloxycarbonyl)methyl)cyclohexane as a colorless liquid was obtained from cyclohexane-1,1-diacetic acid at a yield of 90%. ¹HNMR: δ (ppm) 1.12-1.13 (6H, t, CH₃), 1.3-14 (10H, m, CH₂ of cyclohexane), 2.48 (4H, s, CH₂), 4.0-4.1 (4H, m, CH₂ of ethyl).

### (2) Synthesis of cyclohexane-1,1-diethanol

Synthesis procedure was similar to that described in Example 85(2), and cyclohexane-1,1-diethanol as a colorless viscous liquid from 1,1-bis((ethyloxycarbonyl)methyl)cyclohexane at a yield of 75%. IR spectrum had a strong —OH absorption peak at 3400 cm⁻¹, and had no —CO— absorption peak at about 1700 cm⁻¹. This demonstrated that the reduction reaction was carried out completely.

### (3) Synthesis of 1,1-bis(benzoyloxyethyl)cyclohexane

Synthesis procedure was similar to that described in Example 85(3), and the target product as a colorless viscous liquid was obtained from cyclohexane-1,1-diethanol at a yield of 93%. ¹HNMR: δ (ppm) 1.2-1.4 (6H, m, CH₂ of cyclohexane), 1.4-1.5 (4H, t, CH₂ of cyclohexane), 2.0-2.1 (4H, t, CH₂), 4.1-4.4 (4H, m, CH2 linked to ester radical), δ 7.4-8.1 (10H, m, ArH).

### Example 88

### Synthesis of 1,5-diphenyl-1,5-pentanediol Dibenzoate

### (1) Synthesis of 1,5-diphenyl-1,5-pentanediol

While cooled by an ice-bath and stirred intensely, 0.05 mol 1,5-diphenyl-1,5-pentandione was added dropwise slowly to a mixture of 3 g LiAlH₄ and 100 ml anhydrous tetrahydrofuran. The mixture was heated refluxing for 5 hours, then cooled. Excess LiAlH₄ was decomposed with water. After mixing completely the reaction mixture with ethyl acetate, the mixture was filtered, and the filtrate was dried over anhydrous sodium sulfate. The solvent was removed. Column chromatography gave 1,5-diphenyl-1,5-pentanediol as a white solid, and the yield was 85%. mp: 64-67° C. IR spectrum had a strong —OH absorption peak at 3400 cm⁻¹, and had no —CO— absorption peak at about 1700 cm⁻¹. This demonstrated that the reduction reaction was carried out completely.

### (2) Synthesis of 1,5-diphenyl-1,5-pentanediol Dibenzoate

Synthesis procedure was similar to that described in Example 16, and the target product as a colorless viscous liquid was obtained from 1,5-diphenyl-1,5-pentanediol at a yield of 93%. ¹HNMR: δ (ppm) 1.3-1.5 (2H, s, CH₂), 1.9-2.1 (4H, m, CH₂), 5.94-5.97 (2H, t, CH2 linked to ester radical), 7.2-8.0 (20H, m, ArH).

### Example 89

### Synthesis of 1,5-diphenyl-1,5-pentanediol Dipropionate

Synthesis procedure was similar to that described in Example 4, and the target product as a colorless viscous liquid was obtained from 1,5-diphenyl-1,5-pentanediol and propionyl chloride at a yield of 94%. ¹HNMR: δ (ppm) 1.0-1.1 (6H, m, CH₃), 1.2-1.3 (2H, m, CH₂), 1.7-1.9 (4H, m, CH₂), 2.2-2.3 (4H, m, CH₂ of propyl), 5.6-5.7 (2H, t, CH2 linked to ester radical), 7.2-7.8 (10H, m, ArH).

### Example 90

### Synthesis of bis(2-benzoyloxynaphthyl)methane

The target product was obtained by a synthetic process similar to that described in Example 79. ¹HNMR: δ (ppm) 3.7-3.9 (2H), 6.8-8.1 (22H)

### Example 91

### Synthesis of 3,4-dibutyl-1,6-hexandiol Dibenzoate

The synthetic procedure was identical with that described in Example 2, and 4.3 g product was obtained from 3,4-dibutyl-1,6-hexandiol (4.4 g), benzoyl chloride (3.8 g), pyridine (4.0 g), and tetrahydrofuran (70 ml). ¹HNMR: δ (ppm) 0.8-1.6 (18H), 2.1-2.3 (6H), 4.3-4.5 (4H), 7.4-8.1 (10H).

### Example 92

### Synthesis of 2,2′-biphenyldimethanol Dipivalate

According to the procedure described in Example 4, the target product as a colorless viscous liquid was obtained from 2,2′-biphenyldimethanol and pivaloyl chloride at a yield of 93%. ¹HNMR: δ (ppm) 1.1-1.2 (18H, s, CH₃), 4.84-4.86 (4H, d, CH₂ linked to ester radical), 7.3-7.4 (8H, m, ArH).

### Example 93

### Synthesis of 2,2′-biphenyldimethanol Dibenzoate

### (1) Synthesis of Diethyl 2,2′-biphenyldicarboxylate

To 0.1 mol 2,2′-biphenyl dicarboxylic acid anhydride were added 0.3 mol ethanol, 40 ml toluene, and 0.4 ml concentrated sulfuric acid with stirring. Then the reaction mixture was heated refluxing and the water produced was removed by using a water separator until the amount of the water separated reached theoretical value. Upon the reaction completion, the mixture was neutralized with saturated sodium carbonate solution and extracted with ethyl acetate. The upper layer was separated, washed with saturated saline to neutrality, and dried over anhydrous sodium sulfate. The solvent was removed. Distillation under reduced pressure gave diethyl 2,2′-biphenyldicarboxylate as a colorless liquid, and the yield was 90%.

### (2) Synthesis of 2,2′-biphenyldimethanol

To 3 g LiAlH₄ was added 100 ml anhydrous ethyl ether. While cooling with ice-bath and stirring intensely, 0.05 mol diethyl 2,2′-biphenyldicarboxylate was slowly added dropwise. The reaction mixture was heated refluxing for 5 hours, then cooled. Excess LiAlH₄ was decomposed with water. The mixture was filtered, and the filtrate was extracted with ethyl ether. The extract was dried over anhydrous sodium sulfate. The solvent was removed. Column chromatography gave 2,2′-biphenyldimethanol as a white solid, the yield was 75%, and m.p. was 98-103° C. IR spectrum had a strong absorption peak at 3400 cm⁻¹, and had no absorption peak at about 1700 cm⁻¹. This demonstrated that the reduction reaction was carried out completely.

### (3) Synthesis of 2,2′-biphenyldimethanol Dibenzoate

To 0.03 mol 2,2′-biphenyldimethanol was added 30 ml tetrahydrofuran and 0.09 mol pyridine, then added 0.075 mol benzoyl chloride with stirring. The reaction mixture was heated refluxing for 4 hours, cooled and added 20 ml saturated saline. The mixture was extracted with ethyl acetate, and extract was dried over anhydrous sodium sulfate. The solvent was removed. Column chromatography gave 2,2′-biphenyldimethanol dibenzoate as a colorless viscous liquid, and the yield was 93%. ¹HNMR: δ (ppm) 5.16 (4H, s, CH₂ linked to ester radical), 7.2-8.2 (18H, m, ArH).

### Example 94

### Synthesis of 2,2′-biphenyldimethanol Dipropionate

Synthesis procedure was similar to that described in Example 4, and the target product as a colorless viscous liquid was obtained from 2,2′-biphenyldimethanol and propionyl chloride at a yield of 93%. ¹HNMR: δ (ppm) 1.0-1.1 (6H, t, CH₃), 2.2-2.3 (4H, m, CH₂), 4.8-4.9 (4H, t, CH₂ linked to ester radical), 7.2-7.5 (8H, m, ArH).

### Example 95

### Synthesis of 2,2′-binaphthyldimethanol Dibenzoate

The synthetic procedure was identical with that described in Example 2, and 8.2 g product was obtained from 2,2′-binaphthyldimethanol (4.4 g), benzoyl chloride (4 g), pyridine (4.5 g), and tetrahydrofuran (70 ml). ¹HNMR: δ (ppm) 4.8 (2H), 7.0-8.1 (32H).

### Example 96

### Synthesis of Pentaerythritol Tetrabenzoate

To 4.1 g (0.03 mol) pentaerythritol was added 40 ml tetrahydrofuran, then added 14.5 ml (0.18 mol) pyridine with stirring. To the resulting homogeneous mixture was slowly added 17.4 ml (0.15 mol) benzoyl chloride, and the mixture was stirred for 1 hour at room temperature, then heated refluxing for 6 hours. Upon completing the reaction, 40 ml water was added to dissolve the resulting salt. The mixture was extracted with toluene. Organic phase was separated, washed with saturated saline for two times, dried over anhydrous sodium sulfate, filtered. The solvent was removed to give a white solid. Recrystallization from ethyl acetate gave target product as a white crystal, the yield was 89%. m.p. 95-97° C. ¹HNMR: δ (ppm) 4.77 (s, 8H, CH₂), 7.38-8.02 (m, 20H, ArH).

### Example 97

### Synthesis of 1,2,3-propanetriol Tribenzoate

To 3.7 g (0.04 mol) propanetriol was added 50 ml tetrahydrofuran, then added 14.5 ml (0.18 mol) pyridine with stirring. To the resulting homogeneous mixture was slowly added 17.4 ml (0.15 mol) benzoyl chloride, and the mixture was stirred for 1 hour at room temperature, then heated refluxing for 6 hours. Upon completing the reaction, 40 ml water was added to dissolve the resulting salt. The mixture was extracted with toluene. Organic phase was separated, washed with saturated saline for two times, dried over anhydrous sodium sulfate, filtered. The solvent was removed to give a white solid. Recrystallization from ethyl acetate gave 1,2,3-propanetriol tribenzoate as a white crystal, the yield was 89%, and m.p. was 67-69° C.

¹HNMR δ (ppm):4.73 (d, 4H, CH₂), 5.83 (m, 1H, CH), 7.41-8.07 (m, 15H, aromatic ring H).

### Example 98

### Synthesis of 2,6-dimethyl-3,5-heptandiol di-4-n-butyl-benzoate

To 0.03 mol 2,6-dimethyl-3,5-heptandiol was added 30 ml tetrahydrofuran, then added 0.09 mol pyridine with stirring. To the resulting homogeneous mixture was slowly added 0.075 mol 4-n-butyl-benzoyl chloride, the reaction mixture was heated refluxing for 4 hours, cooled and added 20 ml saturated saline. The mixture was extracted with ethyl acetate, and extract was dried over anhydrous sodium sulfate. The solvent was removed. The product was separated by column chromatography and the yield was 88%. ¹HNMR: δ (ppm): 1.0˜1.1 (18H, m, CH₃), 1.3˜1.4 (4H, m, CH₂), 1.4˜1.5 (4H, m, CH₂), 1.7˜1.8 (2H, m, CH), 2.75˜2.79 (4H, m, CH₂), 2.81˜2.85 (2H, m, CH₂), 5.20˜5.28 (2H, m, CH linked to ester group), 7.2˜8.1 (8H, m, ArH).

### Example 99

### Synthesis of 4,6-nonandiol Dibenzoate

### (1) Synthesis of 4,6-nonandione

Synthesis procedure was similar to that described in Example 33(1). The solvent was removed by distillation to give 0.015 mol product.

### (2) Synthesis of 4,6-nonandiol

Synthesis procedure was similar to that described in Example 33 (3).

### (3) Synthesis of 4,6-nonandiol Dibenzoate

Synthesis procedure was similar to that described in Example 33 (4), and the target product was obtained. ¹HNMR: δ (ppm): 8.0 (10H, ArH), 5.30 (2H, CH), 1.7 (4H, CH₂), 1.4 (4H, CH₂), 2.15 (2H, CH₂), 0.95 (6H, CH₃).

Use of the Polyol Ester Compounds of the Invention

The following examples illustrate the use of the polyol ester compounds according to the present invention in preparation of a catalyst for olefin polymerization. The compounds obtained in examples 8, 9, 15, 50, and 79 were used in preparing a catalyst for olefin polymerization, respectively.

(1) Preparation of the Solid Catalyst Components

To a reactor which was completely replaced with high pure N₂ were added successively 4.8 g magnesium chloride, 95 ml toluene, 4 ml epoxy chloropropane, and 12.5 ml tributyl phosphate. The mixture was heated to 50° C. with stirring and held at the temperature for 2.5 hours to dissolve the solid completely, then added 1.4 g phthalic anhydride and held at the temperature for further one hour. The solution was cooled to below −25° C. and added dropwise 56 ml TiCl₄ over one hour, then heated slowly to 80° C. Solid was precipitated gradually during the heating. To the system were added 6 mmol of polyol ester compounds synthesized in Examples 8, 9, 15, 50, and 79, respectively, and the reaction was held at the temperature with stirring for further one hour. After removing the supernatant, to the residue was added 70 ml toluene and the supernatant was removed again after mixing completely. The washing procedure was repeated twice. The resulting solid precipitate was treated with 60 ml toluene and 40 ml TiCl₄ at 100° C. for 2 hours, and after removing the supernatant, the residue was treated with 60 ml toluene and 40 ml TiCl₄ at 100° C. for 2 hours again. After removing the supernatant, the residue was washed with 60 ml toluene under boiling state for three times, 60 ml hexane under boiling state for two times, 60 ml hexane at normal temperature for two times to yield the solid catalyst components.

(2) Propylene Polymerization Experiments

The catalyst components obtained above were respectively used in the polymerization of propylene. Procedure for the polymerization of propylene was as follow: to a 5 L stainless steel autoclave, which had been replaced with propylene gas completely, were added 2.5 mmol AlEt₃, 0.1 mmol cyclohexylmethyldimethoxysilane (CHMMS), about 10 mg of the solid catalyst component prepared as above, and 1.2 L hydrogen, followed by introduction of 2.3 L liquid propylene. The reactor was heated to 70° C., and the polymerization was performed at that temperature and autogenous pressure for one hour. After the temperature was reduced and the pressure was relieved, PP powder was removed. Polymerization results were summarized in table 1.

TABLE 1
Propylene Polymerization Results of the Solid Catalyst Components
obtained by using the compounds of the present invention

| | | Content of | | Polymerization | Iso- | |
|---|---|---|---|---|---|---|
| | polyol ester | polyol ester | Ti | activity | tacticity | |
| No. | compound | (wt %) | (wt %) | (kgPP/gcat.)* | (%) | MWD |
| Ex.8 | 2,4-pentandiol | 22.1 | 3.1 | 64.2 | 98.6 | 9.7 |
| | di(p-n-butyl-benzoate) | | | | | |
| Ex.9 | 2,4-pentandiol | | | 50.1 | 96.8 | 7.0 |
| | monobenzoate | | | | | |
| | monocinnamate | | | | | |
| Ex.14 | 6-methyl-2,4-heptandiol | 17.6 | 2.7 | 57.9 | 96.8 | 5.3 |
| | dibenzoate | | | | | |
| Ex.50 | 9,9-bis((m-chlorobenz | | | 32.7 | 98.5 | 7.1 |
| | oyloxy)-methyl)flurene | | | | | |
| Ex.79 | 2,3-diisopropyl-1,4- | | | 31.7 | 96.9 | 6.6 |
| | butandiol dibenzoate | | | | | |

*Polymerization activity: kilograms of the polymer obtained per gram solid catalyst component.

Although the present invention has been described in connection with embodiments and examples, further modifications and alternative embodiments of various aspects of the invention will be apparent to those skilled in the art in view of this description. Accordingly, this description is to be constructed as illustrative only and is for the purpose of teaching the general manner of carrying out the invention. Additionally, all cited documents are wholly incorporated into this description by reference.

1. Polyol ester compounds, having general formula (I):
R₁CO—O—CR₃R₄-A-CR₅R₆—O—CO—R₂ (I)
wherein, R₁ and R₂ groups, which may be identical or different, can be selected from the group consisting of C₁-C₂₀ substituted or unsubstituted, linear or branched alkyl, cycloalkyl, aryl, alkaryl, aralkyl, alkenyl, fused aryl, alkoxycarbonyl, R₃-R₆ groups, which may be identical or different, can be selected from the group consisting of hydrogen, halogen or C₁-C₂₀ substituted or unsubstituted, linear or branched alkyl, cycloalkyl, aryl, alkaryl, aralkyl, alkenyl, fused aryl, R₁-R₆ groups optionally contain one or more halogen atoms replacing carbon, hydrogen or the both, two or more of R₃-R₆ groups can be linked to form saturated or unsaturated monocyclic or polycyclic ring;
A is a bivalent linking group with chain length between two free radicals being 1-4 atoms, wherein said bivalent linking group is selected from the group consisting of aliphatic, alicyclic and aromatic bivalent radicals, and can carry C₁-C₂₀ substituents selected from the group consisting of linear or branched alkyl, cycloalkyl, aryl, alkaryl, aralkyl, alkenyl, fused aryl; and two or more said substituents on the linking group can be linked to form saturated or unsaturated monocyclic or polycyclic ring;
with the proviso that:
when A is —R¹CR²—, in which R¹ and R² are independently each other selected from hydrogen or C₁-C₂₀ hydrocarbyl group which may be unsubstituted or substituted by one or more halogen atoms, then
(i) R¹, R², R₃, R₄, R₅ and R₆ groups are not simultaneously hydrogen or halogen;
(ii) when R₃, R₄, R₅ and R₆ groups are simultaneously hydrogen, R¹ and R² are selected from the group consisting of linear or branched alkyl and cycloalkyl having 3 to 20 carbon atoms, and R¹, R², R₃, R₄, R₅ and R₆ groups are not linked each other to form a ring with the exception that R¹ and R² can together form a fluorene ring, and in this case at least one of R₁ and R₂ are not phenyl;
(iii) among the two groups R₃ and R₄ which attach to same one carbon atom as well as among the two groups R₅ and R₆ which attach to another carbon atom, when one is hydrogen atom, the other is not hydrogen or halogen atom, and
(a) when the two groups other than hydrogen or halogen are all methyl, and R¹ and R² are all hydrogen or one is hydrogen and the other is methyl, then R₁ and R₂ are not identical;
(b) when one of the two groups other than hydrogen or halogen is methyl, then the other is not n-butyl or iso-butyl, or R¹ and R² are not simultaneously hydrogen;
when A is —R¹CR²—R³CR⁴—, in which R¹-R⁴ are independently each other selected from hydrogen or C₁-C₂₀ hydrocarbyl group which may be unsubstituted or substituted by one or more halogen atoms, then
(i) groups R₃-R₆ and R¹-R⁴ cannot linked each other to form a ring;
(ii) at least two groups of R₃-R₆ and R¹-R⁴ are other than hydrogen or halogen; and
(iii) when one of the two groups R₃ and R₄ and one of the two groups R₅ and R₆ are hydrogen or halogen, and R¹-R⁴ are all hydrogen, the other groups cannot be simultaneously methyl;
when A is —R¹CR²—R³CR⁴—R⁵CR⁶—, in which R¹-R⁶ are independently each other selected from hydrogen or C₁-C₂₀ hydrocarbyl group which may be unsubstituted or substituted by one or more halogen atoms, then
(i) at least two groups of R₃R₆ and R¹-R⁶ are not hydrogen;
(ii) when only two groups of R₃-R₆ and R¹-R⁶ are hydrogen, the other groups cannot all be methyl or ethyl; and
(iii) groups R₃-R₆ and R¹-R⁶ cannot be linked each other to form a ring with the exception of forming a naphthalene ring, and in the later case, R₁ and R₂ do not contain halogen atom;
when A is —R¹CR²—R³CR⁴—R⁵CR⁶—R⁷CR⁸—, in which R¹-R⁸ are independently each other selected from hydrogen or C₁-C₂₀ hydrocarbyl group which may be unsubstituted or substituted by one or more halogen atoms, then
(i) groups R₃-R₆ and R¹-R⁸ cannot be linked each other to form a ring; and
(ii) when two groups of R₃-R₆ and R¹-R⁸ are methyl, the other cannot simultaneously be hydrogen.

2. The polyol ester compounds of claim 1, being 1,3-diol ester compounds of general formula (III): 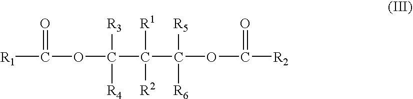
wherein R₃-R₆ are selected from the group consisting of hydrogen, and ethyl, propyl, isopropyl, butyl, tert-butyl, and phenyl, with these groups being unsubstituted or substituted by halogen; R¹ and R², which may be identical or different, can be selected from the group consisting of hydrogen, and methyl, ethyl, propyl, iso-propyl, butyl, tert-butyl, allyl, and phenyl or C₁₋₆-alkylphenyl, with these groups being unsubstituted or substituted by halogen; and at least one of R₁ and R₂ are benzene ring-containing group.

3. The polyol ester compounds of claim 2, wherein at least one group of R₁ and R₂ is phenyl or phenyl substituted by halogen or alkyl having 1 to 20 carbon atoms.

4. The polyol ester compounds of claim 2, wherein both R₁ and R₂ are phenyl or phenyl substituted by halogen or alkyl having 1 to 20 carbon atoms.

5. The polyol ester compounds of claim 2, wherein among the two groups R₃ and R₄ which attach to same one carbon atom as well as among the two groups R₅ and R₆ which attach to another carbon atom, when one is hydrogen atom, the other is selected from the group consisting of ethyl, propyl, iso-propyl, butyl, tert-butyl and phenyl, with these groups being unsubstituted or substituted by halogen; R¹ and R², which may be identical or different, can be selected from the group consisting of hydrogen, and methyl, ethyl, propyl, iso-propyl, butyl, tert-butyl, allyl, and phenyl, with these groups being unsubstituted or substituted by halogen; both R₁ and R₂ are phenyl or phenyl substituted by halogen or alkyl having 1 to 20 carbon atoms.

6. The polyol ester compounds of claim 1, being 1,4-diol ester compounds of a general formula (IV): 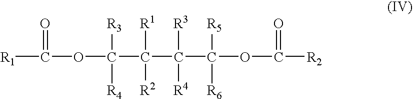
wherein R₁-R₆ have the meanings as defined in general formula (I), R¹-R⁴ are independently each other hydrogen or C₁-C₂₀ hydrocarbyl group which may be unsubstituted or substituted by one or more halogen atoms, with the proviso that R¹-R⁴ are not hydrogen simultaneously, R¹-R⁴ and R₃-R₆ can not be linked each other to form a ring, and among the two groups R₃ and R₄ as well as among the two groups R₅ and R₆, there are, respectively, at least one being hydrogen or halogen.

7. The polyol ester compounds of claim 6, wherein among the two groups R₃ and R₄ as well as among the two groups R₅ and R₆, one is hydrogen atom, and the other is methyl, ethyl, propyl, iso-propyl, butyl, tert-butyl, and phenyl or halophenyl; R¹-R⁴, which may be identical or different, can be selected from the group consisting of hydrogen, methyl, ethyl, propyl, iso-propyl, butyl, tert-butyl, and phenyl or halophenyl; and at least one of R₁ and R₂ are benzene ring-containing group.

8. The polyol ester compounds of claim 7, wherein at least one group of R₁ and R₂ is phenyl or phenyl substituted by halogen or alkyl having 1 to 20 carbon atoms.

9. The polyol ester compounds of claim 7, wherein both R₁ and R₂ are phenyl or phenyl substituted by halogen or alkyl having 1 to 20 carbon atoms.

10. The polyol ester compounds of claim 6, wherein among the two groups R₃ and R₄ as well as among the two groups R₅ and R₆, one is hydrogen atom, and the other is methyl, ethyl, propyl, iso-propyl, butyl, tert-butyl, and phenyl or halophenyl; R¹-R⁴, which may be identical or different, can be selected from the group consisting of hydrogen, methyl, ethyl, propyl, iso-propyl, butyl, tert-butyl, and phenyl or halophenyl; and both R₁ and R₂ are phenyl or phenyl substituted by halogen or alkyl having 1 to 20 carbon atoms.

11. The polyol ester compounds of claim 1, being 1,5-diol ester compound of a general formula (V): 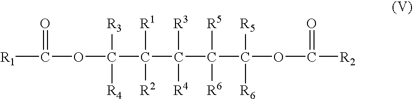
wherein R₁-R₆ have the meanings as defined in general formula (I), R¹-R⁶ are independently each other hydrogen or C₁-C₂₀ hydrocarbyl group which may be unsubstituted or substituted by one or more halogen atoms, with the proviso that at least two groups of R₃-R₆ and R¹-R⁶ are not hydrogen; when only two groups of R₃-R₆ and R¹-R⁶ are hydrogen, the other groups cannot all be methyl or ethyl; groups R₃-R₆ and R¹-R⁶ cannot be linked each other to form a ring with the exception of forming a naphthalene ring, and in the later case, R₁ and R₂ do not contain halogen atom.

12. The polyol ester compounds of claim 11, wherein at least one group of R₁ and R₂ is phenyl or phenyl substituted by halogen or alkyl having 1 to 20 carbon atoms.

13. The polyol ester compounds of claim 11, wherein both R₁ and R₂ are phenyl or phenyl substituted by halogen or alkyl having 1 to 20 carbon atoms.

14. The polyol ester compounds of claim 1, being 1,6-diol ester compound of a general formula (VI): 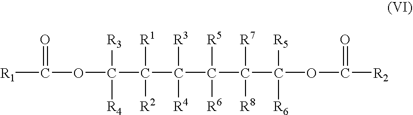
wherein R₁-R₆ have the meanings as defined in general formula (I), R¹-R⁸ are independently each other hydrogen or C₁-C₂₀ hydrocarbyl group which may be unsubstituted or substituted by one or more halogen atoms, with the proviso that groups R₃-R₆ and R¹-R⁸ cannot be linked each other to form a ring; and when two groups of R₃-R₆ and R¹-R⁸ are methyl, the other cannot all be hydrogen.

15. The polyol ester compounds of claim 14, wherein at least one group of R₁ and R₂ is phenyl or phenyl substituted by halogen or alkyl having 1 to 20 carbon atoms.

16. The polyol ester compounds of claim 14, wherein both R₁ and R₂ are phenyl or phenyl substituted by halogen or alkyl having 1 to 20 carbon atoms.

17. The polyol ester compound of claim 1, being compounds of general formula (VII): 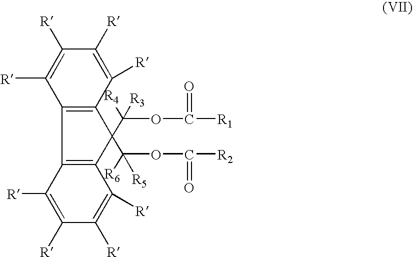
wherein R₁-R₆ are as defined in general formula (I), R′, which can be identical or different, represents hydrogen, halogen atom, linear or branched C₁-C₂₀ alkyl, C₃-C₂₀ cycloalkyl, C₆-C₂₀ aryl, C₇-C₂₀ alkaryl or C₇-C₂₀ aralkyl group, with the proviso that R₁ and R₂ cannon be phenyl simultaneously.

18. The polyol ester compound of claim 1, which is selected from the group consisting of:
2,4-pentanediol di(m-chlorobenzoate)
2,4-pentanediol di(o-bromobenzoate)
2,4-pentanediol di(p-methylbenzoate)
2,4-pentanediol di(p-tert-butylbenzoate)
2,4-pentanediol di(p-butylbenzoate)
2,4-pentanediol monobenzoate monocinnamate
2,4-pentanediol dicinnamate
heptan-6-ene-2,4-diol dibenzoate
3,5-heptandiol dibenzoate
2,6-dimethyl-3,5-heptandiol dibenzoate
6-methyl-2,4-heptanediol dibenzoate
6-methyl-2,4-heptanediol di(p-chlorobenzoate)
6-methyl-2,4-heptanediol di(p-methylbenzoate)
6-methyl-2,4-heptanediol di(m-methylbenzoate)
6-methyl-2,4-heptanediol dipivalate
3-methyl-2,4-pentanediol di(p-chlorobenzoate)
3-methyl-2,4-pentanediol di(p-methylbenzoate)
3-butyl-2,4-pentanediol di(p-methylbenzoate)
3-methyl-2,4-pentanediol di(p-tert-butylbenzoate)
3-methyl-2,4-pentanediol monobenzonate monocinnamate
3,3-dimethyl-2,4-pentandiol dibenzoate
3,3-dimethyl-2,4-pentandiol monobenzonate monocinnamate
3-ethyl-2,4-pentandiol dibenzoate
3-butyl-2,4-pentandiol dibenzoate
3-allyl-2,4-pentandiol dibenzoate
4-methyl-3,5-heptandiol dibenzoate
2-ethyl-1,3-hexandiol dibenzoate
2,2,4-trimethyl-1,3-pentandiol dibenzoate
4-methyl-3,5-octandiol dibenzoate
5-methyl-4,6-nonandiol dibenzoate
2-methyl-1,3-diphenyl-1,3-propylene-glycol dibenzoate
1,3-diphenyl-1,3-propylene-glycol dipropionate
2-methyl-1,3-diphenyl-1,3-propylene-glycol dipropionate
2-methyl 1,3-diphenyl-1,3-propylene-glycol diacetate
2,2-dimethyl-1,3-diphenyl-1,3-propylene-glycol dibenzoate
2,2-dimethyl-1,3-diphenyl-1,3-propylene-glycol dipropionate
2-methyl-1-phenyl-1,3-butandiol dibenzoate
2-methyl-1-phenyl-1,3-butandiol dipivalate
heptan-6-ene-2,4-diol dipivalate
2,2,4,6,6-pentamethyl-3,5-hexandiol dibenzoate
1,3-di-tert-butyl-2-ethyl-1,3-propylene-glycol dibenzoate
1,3-diphenyl-1,3-propylene-glycol diacetate
2-(2-furyl)-2-methyl-1,3-butandiol dibenzoate
1,1-di(acryloyloxymethyl)-3-cyclohexene
2-isoamyl-2-isopropyl-1,3-propylene-glycol dibenzoate
2-isoamyl-2-isopropyl-1,3-propylene-glycol di(p-chlorobenzoate)
2-isoamyl-2-isopropyl-1,3-propylene-glycol di(m-chlorobenzoate)
2-isoamyl-2-isopropyl-1,3-propylene-glycol di(p-methoxybenzoate)
2-isoamyl-2-isopropyl-1,3-propylene-glycol di(p-methylbenzoate)
2-isoamyl-2-isopropyl-1,3-propylene-glycol monobenzoate monopropionate
2-isoamyl-2-isopropyl-1,3-propylene-glycol dipropionate
2-isoamyl-2-isopropyl-1,3-propylene-glycol diacrylate
2-isoamyl-2-isopropyl-1,3-propylene-glycol dicinnamate
2,2-diisobutyl-1,3-propylene-glycol dibenzoate
2-isoamyl-2-isopropyl-1,3-propylene-glycol 2,2′-biphenyl dicarboxylate
2-isoamyl-2-isopropyl-1,3-propylene-glycol phthalate
1,3-diisopropyl-1,3-propylene-glycol di(4-butylbenzoate)
3-methyl-1-trifluoromethyl-2,4-pentandiol dibenzoate
1,1,1-trifluoro-3-methyl-2,4-pentandiol dibenzoate
4,4,4-trifluoro-1-(2-naphthyl)-1,3-butandiol dibenzoate
2-ethyl-2-methyl-1,3-propylene-glycol dipropylformate
2,4-pentanediol di(p-fluoromethylbenzoate)
4,6-nonandiol dibenzoate
2,4-pentandiol di(2-furancarboxylate)
2-amino-1-phenyl-1,3-propylene-glycol dibenzoate
2,2-dimethyl-1,3-propylene-glycol dibenzoate
3-butyl-3-methyl-2,4-pentandiol dibenzoate
3,6-dimethyl-2,4-heptandiol dibenzoate
2,2,6,6-tetramethyl-3,5-heptandiol dibenzoate
2,3-diisopropyl-1,4-butandiol dibenzoate
2,3-dimethyl-1,4-butandiol dibenzoate
2,3-diethyl-1,4-butandiol dibenzoate
2,3-dibutyl-1,4-butandiol dibenzoate
2,3-diisopropyl-1,4-butandiol dibutyrate
2,5-hexandiol dicinnamate
2,5-dimethyl-2,5-hexandiol dibenzoate
2,5-dimethyl-2,5-hexandiol dipropionate
2,5-dimethyl-hexa-3-yne-2,5-diol dibenzoate
hexa-3-yne-2,5-diol dibenzoate (T)
hexa-3-yne-2,5-diol dibenzoate (S)
hexa-3-yne-2,5-diol di(2-furancarboxylate)
1,1-bis(benzoyloxyethyl)cyclohexane
2,2-dimethyl-1,5-pentanediol dibenzoate
1,5-diphenyl-1,5-pentanediol dibenzoate
1,5-diphenyl-1,5-pentanediol dipropionate
2,6-dimethyl-2,6-heptanediol dibenzoate
bis(2-benzoyloxynaphthyl)methane
3,4-dibutyl-1,6-hexandiol dibenzoate
2,2′-biphenyldimethanol dipivalate
2,2′-biphenyldimethanol dibenzoate
2,2′-biphenyldimethanol dipropionate
2,2′-binaphthyldimethanol dibenzoate
9,9-bis((m-methoxybenzoyloxy)methyl)fluorene
9,9-bis((m-chlorobenzoyloxy)methyl)fluorene
9,9-bis((p-chlorobenzoyloxy)methyl)fluorene
9,9-bis(cinnamoyloxymethyl)fluorene
9-(benzoyloxymethyl)-9-(propionyloxymethyl)fluorene
9,9-bis(propionyloxymethyl)fluorene
9,9-bis(acryloyloxymethyl)fluorene
9,9-bis(pivalyloxymethyl)fluorene

19. A process for preparing a polyol ester compound according to claim 1, comprising esterification of a polyol compound of general formula (VIII)
HO—CR₃R₄-A-CR₅R₆—OH (VIII)
wherein A, R₃-R₆ are as defined in the general formula (I).

20. A process comprising using a polyol ester compound according claim 1 in preparation of a catalyst for olefin polymerization.
